(19) United States
(12) Patent Application Publication
(10) Pub. No.: US 2005/0106563 A1
(43) Pub. Date: May 19, 2005

(54) Epitope profiles of SARS coronavirus

(75) Inventors: Jen-Pin Huang, Sindian City (TW); Lee-Hsuan Chen, Taipei City (TW)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Genesis Biotech Inc.

(21) Appl. No.: 10/936,237

(22) Filed: Sep. 8, 2004

Related U.S. Application Data
(60) Provisional application No. 60/500,702, filed on Sep. 8, 2003.

(51) Int. Cl. C12Q 1/70; C07K 9/00

(52) U.S. Cl. 435/5; 530/326; 530/322

(57) ABSTRACT

The present invention relates to the identification of SARS corona virus epitopes specific to anti-SARS corona virus antibodies found in the sera of SARS patients. Epitope profiles can be used to detect and characterize SARS-CoV infection. The epitope polypeptides can also be used as immunogenic peptides to create polyclonal and/or monoclonal antibodies against SARS coronavirus.

### DESCRIPTION OF THE INVENTION

This application claims the benefit of U.S. Provisional Application No. 60/500,702, filed Sep. 8, 2003, which is incorporated herein by reference.

### FIELD OF THE INVENTION

The invention relates to the identification of epitopes of SARS-associated coronavirus, and more specifically, a time-dependent epitope profile of the SARS coronavirus infected patients.

### BACKGROUND OF THE INVENTION

In 2003, a severe febrile respiratory disease was reported in China, Vietnam, Canada, Hong Kong and Taiwan. Within months of initial reports, the illness had spread from infected persons to healthcare workers and household members. The syndrome was named “severe acute respiratory syndrome” (SARS) in March 2003. Also, in March 2003, a novel coronavirus from SARS patients was isolated (SARS-CoV).

Most coronaviruses cause disease in only one host species. All known coronaviruses are found in three different groups. Two of them can infect mammalian animals and one can infect poultry. SARS-COV was not similar to other human coronaviruses (HCoV-229E and HCoV-OC43), and researchers suggested that it be classified into a new group.

This type of coronavirus has a genome of more than 29,700 nucleotides, and it has a complex two-step replication mechanism. Generally, for viral replications, many RNA virus genomes contain a gene that is translated by the host's system to produce all viral proteins. This gene has been called the replicase gene. The structural proteins of coronaviruses (spike (S or E2), small envelope (sE or E), matrix (M), and nucleocapsid (N)) function during host cell entry and virion morphogenesis and release. The SARS-CoV has several small open reading frames (ORFs) that are found between the S and sE genes and between the M and N genes. The functions of these small ORFs are still unknown, and the complete mechanism of SARS-CoV is yet to be determined.

The SARS-CoV's complete genomic sequences and encoded protein sequences have been available on the web at GeneBank.

### SUMMARY OF THE INVENTION

The present invention provides the relevant epitopes of the SARS-COV and various epitope profiles, i.e. a synopsis of epitopes and anti-SARS-CoV sera cross reactions. The invention also provides peptide immunogens used to produce polyclonal and monoclonal antibodies against SARS-CoV. The availability of SARS-CoV epitope sequence profiles would be useful in controlling the disease by making it possible to develop diagnostic tests, vaccines, and antiviral agents.

Specifically, the invention provides polypeptide(s), which encompass epitopes specific to anti-SARS-CoV sera (sera containing antibodies against SARS-CoV), comprising any one or more of the amino acid sequence(s) selected from SEQ ID NO: 40, SEQ ID NO: 42, SEQ ID NO: 46, SEQ ID NO: 47, SEQ ID NO: 50, SEQ ID NO: 54, SEQ ID NO: 55, SEQ ID NO: 57, SEQ ID NO: 58, SEQ ID NO: 59, SEQ ID NO: 60, SEQ ID NO: 61, SEQ ID NO: 62, SEQ ID NO: 65, SEQ ID NO: 69, SEQ ID NO: 70, SEQ ID NO: 71, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 112, SEQ ID NO: 139, SEQ ID NO: 140, SEQ ID NO: 156, SEQ ID NO: 183, and SEQ ID NO: 187 and degenerate variants thereof. These epitopes are also referred to as the SARS-COV specific epitopes.

The invention further provides polypeptides which encompass epitopes not specific to anti-SARS-CoV sera comprising any one or more of the amino acid sequences selected from SEQ ID NO: 11, SEQ ID NO: 12, SEQ ID NO: 25, SEQ ID NO: 64, SEQ ID NO: 67, SEQ ID NO: 81, SEQ ID NO: 88, and SEQ ID NO: 118 and degenerate variants thereof. These epitopes are referred to as the SARS-COV non-specific epitopes.

Also provided are polypeptides which encompass inflammation epitopes of SARS-COV (SARS-COV specific and non-specific epitopes having stronger antibody binding activities (AT>1) during hospitalization than in post-hospitalization period), comprising any one or more of the amino acid sequences of SEQ ID NO: 11, SEQ ID NO: 12, SEQ ID NO: 25, SEQ ID NO: 40, SEQ ID NO: 42, SEQ ID NO: 46, SEQ ID NO: 49, SEQ ID NO: 50, SEQ ID NO: 51, SEQ ID NO: 54, SEQ ID NO: 55, SEQ ID NO: 57, SEQ ID NO: 58, SEQ ID NO: 59, SEQ ID NO: 60, SEQ ID NO: 61, SEQ ID NO: 62, SEQ ID NO: 64, SEQ ID NO: 65, SEQ ID NO: 67, SEQ ID NO: 69, SEQ ID NO: 70, SEQ ID NO: 71, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 88, and SEQ ID NO: 140 and degenerate variants thereof.

Moreover, the invention provides for novel polypeptides, used as a control in the assays, with sequences of any one or more of SEQ ID NO: 1, SEQ ID NO: 2, SEQ ID NO: 3, SEQ ID NO: 4, SEQ ID NO: 5, and SEQ ID NO: 6.

In addition, the invention provides an immunogenic composition that comprises one or more polypeptides comprising amino acid sequences selected from SEQ ID NO: 7, SEQ ID NO: 11, SEQ ID NO: 12, SEQ ID NO: 17, SEQ ID NO: 19, SEQ ID NO: 21, SEQ ID NO: 25, SEQ ID NO: 27, SEQ ID NO:36, SEQ ID NO: 37, SEQ ID NO: 38, SEQ ID NO: 44, SEQ ID NO: 51, SEQ ID NO: 53, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 67, SEQ ID NO: 68, SEQ ID NO: 69, SEQ ID NO: 70, SEQ ID NO: 76, SEQ ID NO: 82, SEQ ID NO: 83, SEQ ID NO: 84, SEQ ID NO: 90, SEQ ID NO: 95, SEQ ID NO: 97, SEQ ID NO: 98, and SEQ ID NO: 99 and degenerate variants thereof.

The polypeptides described by the invention may be in a linear or branched form.

The invention further provides an apparatus bearing one or more polypeptide(s) comprising amino acid sequence(s) selected from SEQ ID NO: 7-SEQ ID NO: 195 and degenerate variants thereof. The invention also provides for an apparatus bearing one or more polypeptide(s) comprising the amino acid sequence(s) selected from SEQ ID NO: 44-SEQ ID NO: 62 (polypeptides related to the N1 protein); or in the alternative SEQ ID NO: 63-SEQ ID NO: 82 (polypeptides related to the N2 protein); SEQ ID NO: 124-SEQ ID NO: 142 (polypeptides related to the X2 protein); SEQ ID NO: 83-SEQ ID NO: 89 and SEQ ID NO: 112-SEQ ID NO: 123 (polypeptides related to the M protein); or SEQ ID NO: 7-SEQ ID NO: 43 (polypeptides related to the S protein).

In addition, the invention provides an apparatus bearing one or more polypeptide(s) comprising the amino acid sequence(s) selected from, SEQ ID NO: 59, SEQ ID NO: 60, and SEQ ID NO: 79; or in the alternative SEQ ID NO: 59, SEQ ID NO: 60, SEQ ID NO: 77, SEQ ID NO: 79, SEQ ID NO: 112, SEQ ID NO: 139, SEQ ID NO: 140, and SEQ ID NO: 183, and SEQ ID NO: 187 (the immediate early epitopes identified during 1-6 days of hospitalization); or SEQ ID NO: 71 (the early epitope identified in the following 7-29 days after the initial 1-6 days).

It is to be understood that both the foregoing general description and the following detailed description are exemplary and explanatory only and are not restrictive of the invention, as claimed.

The accompanying drawings, which are incorporated herein and constitute a part of this specification, illustrate one (several) embodiment(s) of the invention and together with the description, serve to explain the principles of the invention.

### BRIEF DESCRIPTION OF THE DRAWINGS

FIG. 1 is a pictorial diagram of the SARS-COV epitope related polypeptides. The polypeptides were synthesized in the form of multiple antigenic peptide (eight branched) or linear polypeptide, as listed on Table 1. These polypeptides span the SARS-COV spike (S1: aa 421-˜520, S2: 1021˜1120, 1116˜1200), nucleocapsid (N1: 70˜169, N2: 300˜399), matrix (M: 1˜20, 61˜85, 95˜203), small envelope (sE: 1˜20, 16˜76), protein X1 (1˜40, 93˜102), protein X2 (24˜98, 119˜149), protein X3 (1˜63), protein X4 (70˜119), and protein X5 (1˜84) regions. The adjacent polypeptides are overlapping in 4˜6 amino acids in continuous sequences.

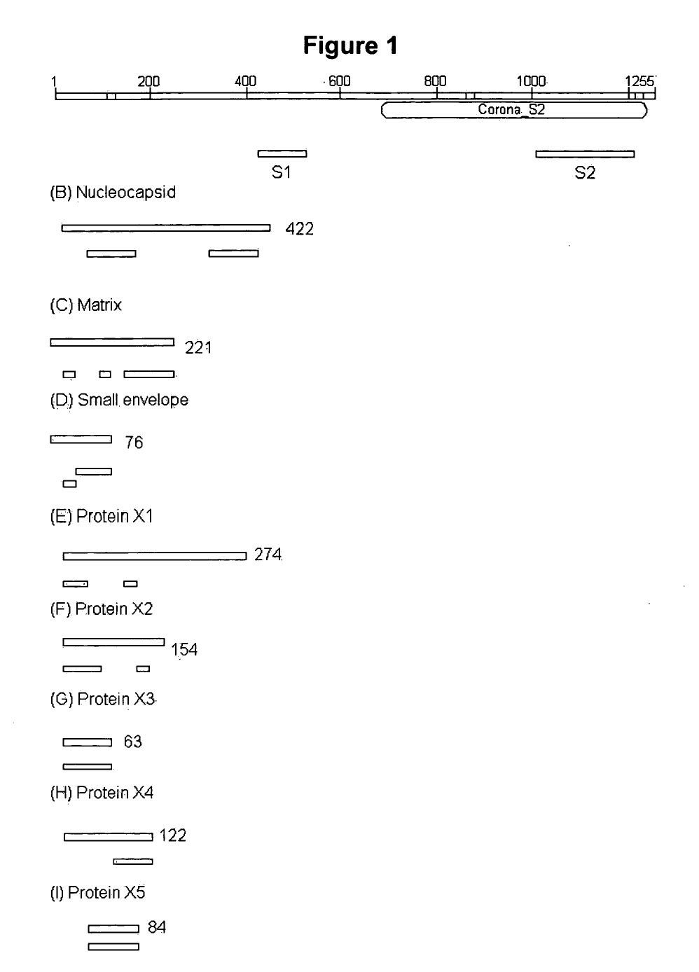

FIG. 2 is the ELISA result of serum from five groups of subjects (group 0 consists of Genesis employees outside of hospital, group 1 consists of hospital employees, group 2 consists of SARS suspected patients, group 3 consists of SARS probable patients, and group 4 consists of recovering patients from groups 2 and 3) using the control polypeptides.

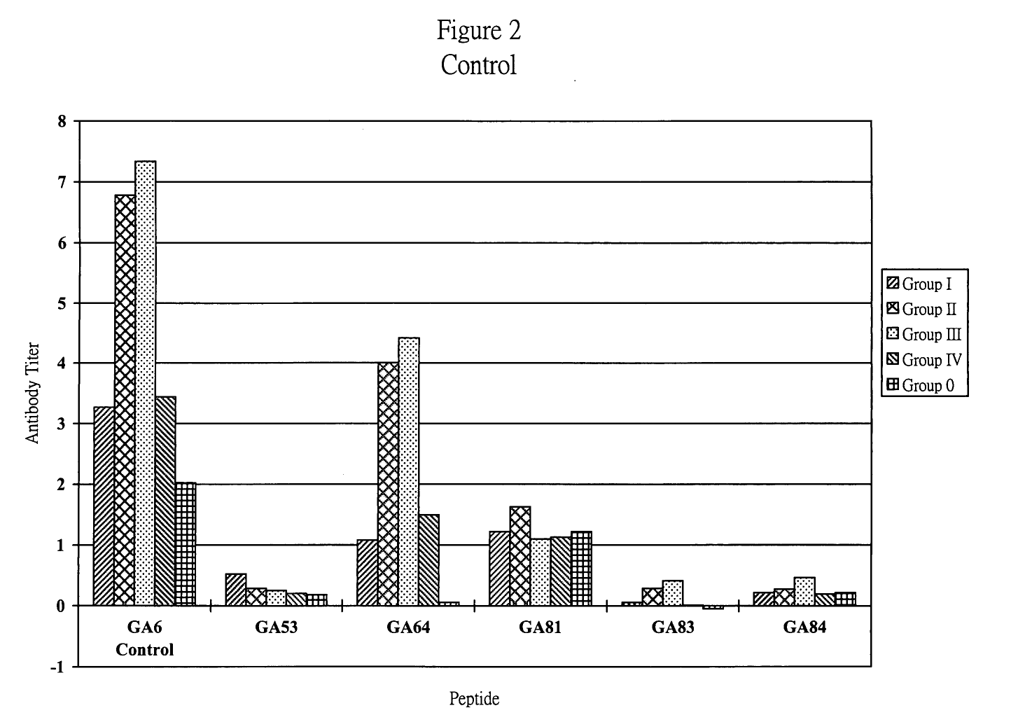

FIG. 3 is the ELISA result of serum from the five groups of subjects using the designated SARS-COV S1 polypeptides.

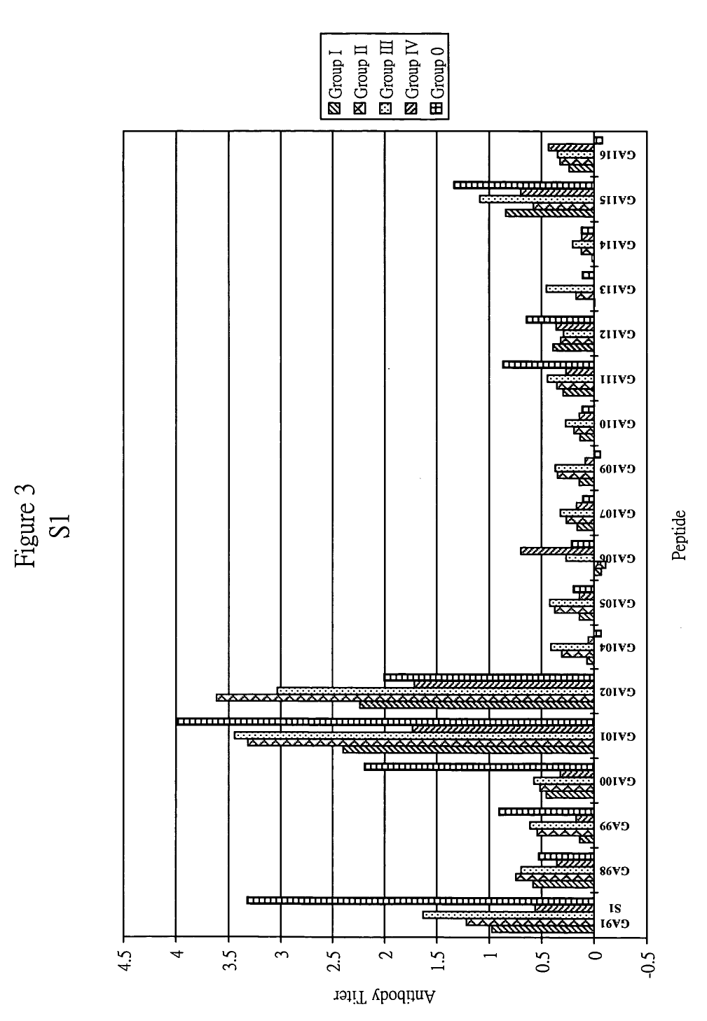

FIG. 4 is the ELISA result of serum from the five groups of subjects using the designated SARS-COV S2 polypeptides.

FIG. 5 is the ELISA result of serum from the five groups of subjects using the designated SARS-COV N1 polypeptides.

FIG. 6 is the ELISA result of serum from the five groups of subjects using the designated SARS-CoV N2 polypeptides.

FIG. 7 is the ELISA result of serum from the five groups of subjects using the designated SARS-COV M polypeptides.

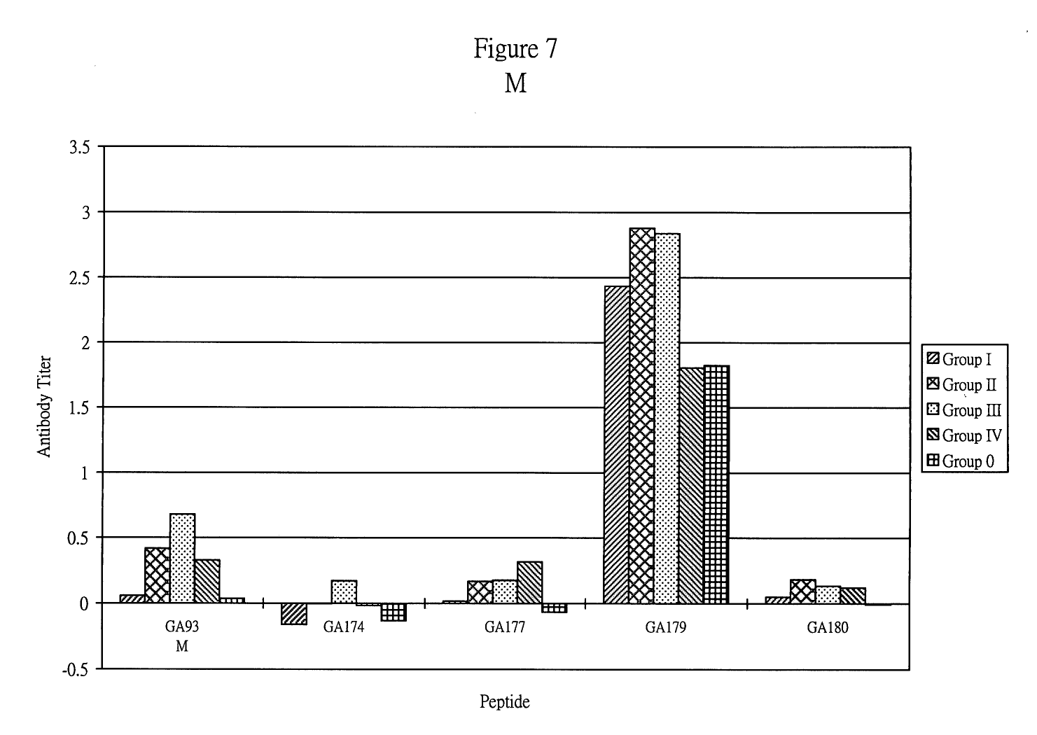

FIG. 8 is the ELISA result of serum from the five groups of subjects using additional designated SARS-CoV M polypeptides.

FIG. 9 is the ELISA result of serum from the five groups of subjects using the designated SARS-COV sE polypeptides.

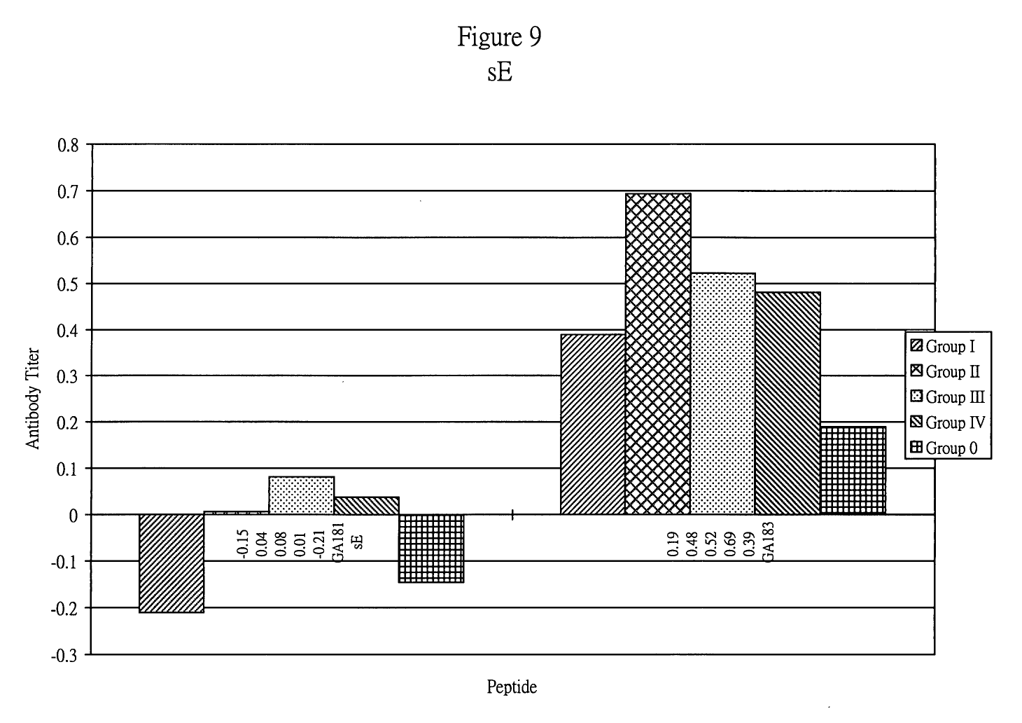

FIG. 10 is the ELISA result of serum from the five groups of subjects using additional designated SARS-CoV sE polypeptides.

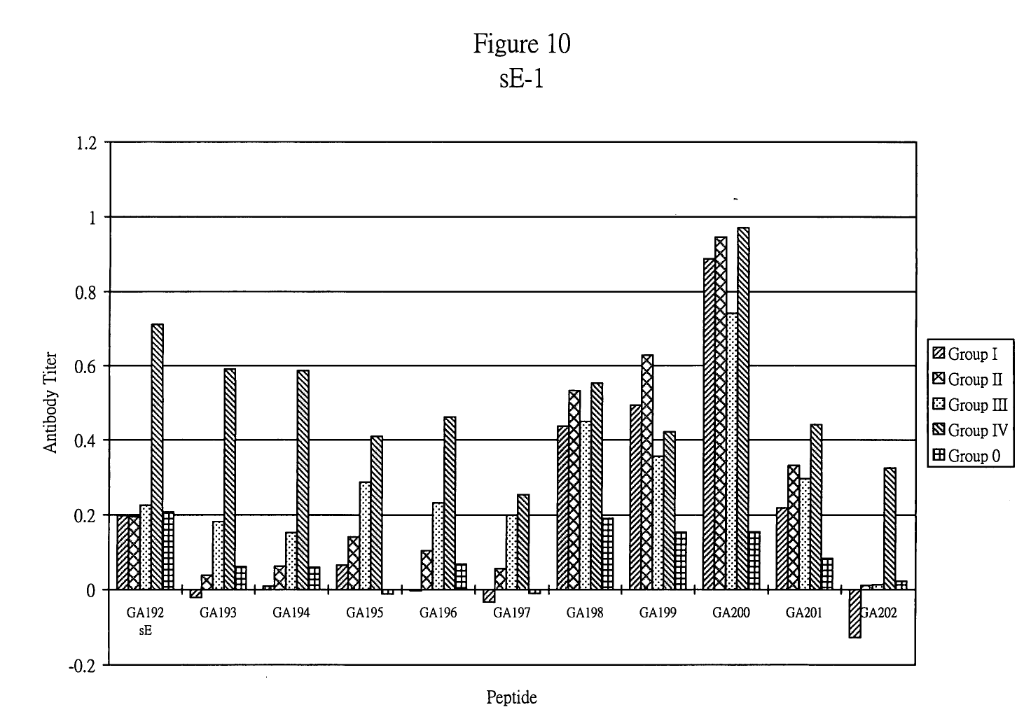

FIG. 11 is the ELISA result of serum from the five groups of subjects using the designated SARS-COV X1 polypeptides.

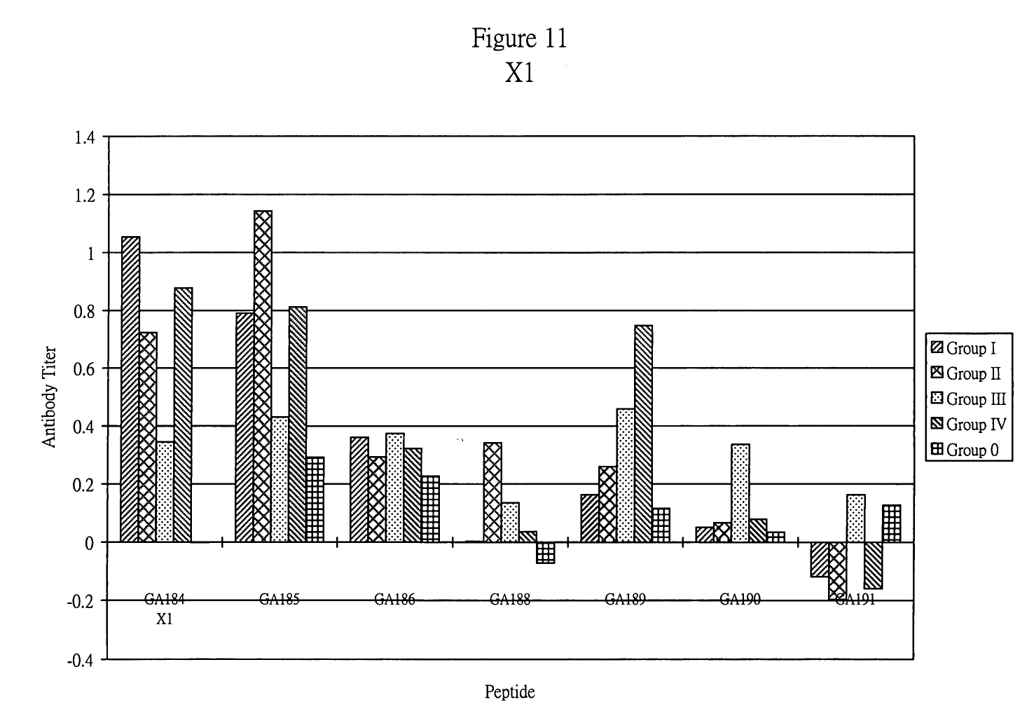

FIG. 12 is the ELISA result of serum from the five groups of subjects using the designated SARS-COV X2 polypeptides.

FIG. 13 is the ELISA result of serum from the five groups of subjects using the designated SARS-COV X3 polypeptides.

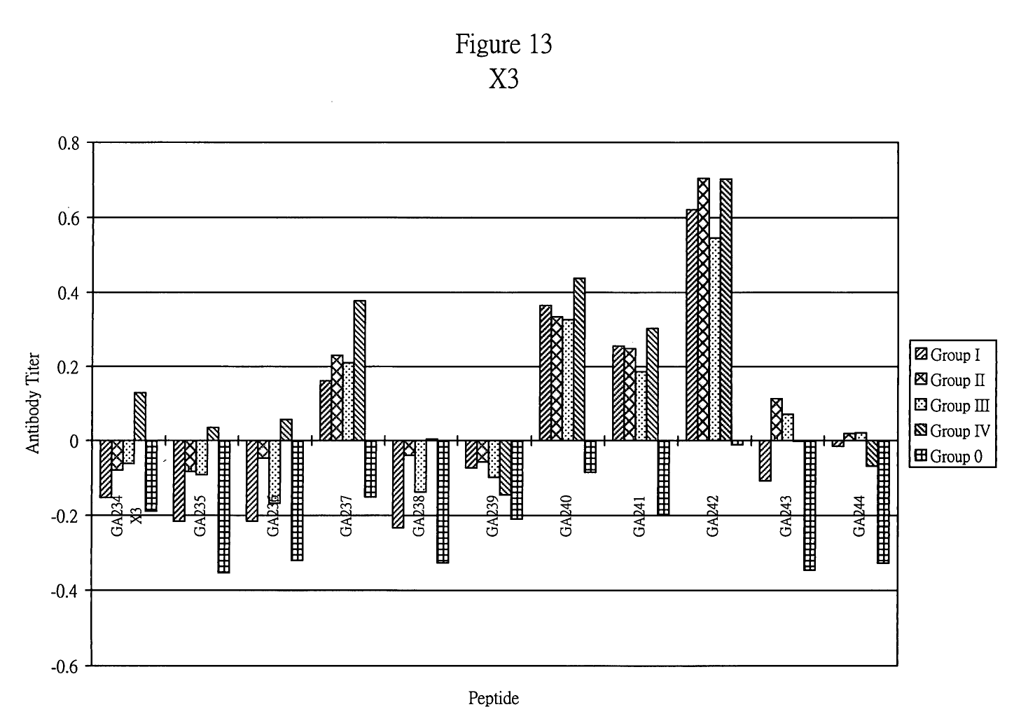

FIG. 14 is the ELISA result of serum from the five groups of subjects using the designated SARS-COV X4 polypeptides.

FIG. 15 is the ELISA result of serum from the five groups of subjects using the designated SARS-CoV X5 polypeptides.

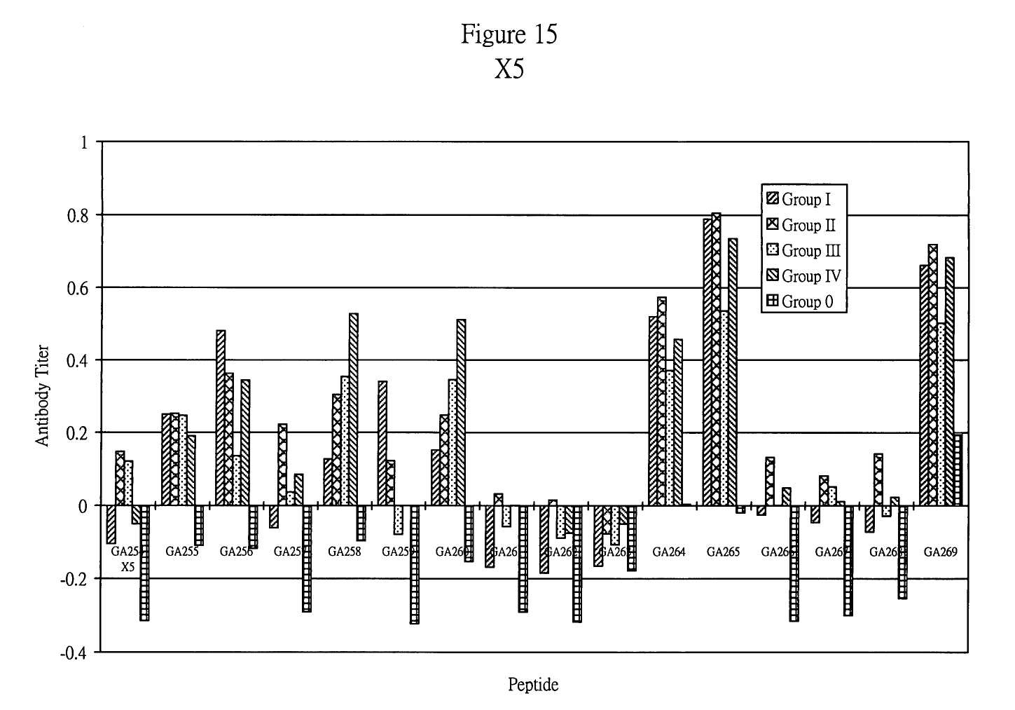

FIG. 16 is the epitope profiles of control subjects' sera IgG immune response in view of epitopes N1: GA151, GA152 and N2: GA162, GA168, GA170.

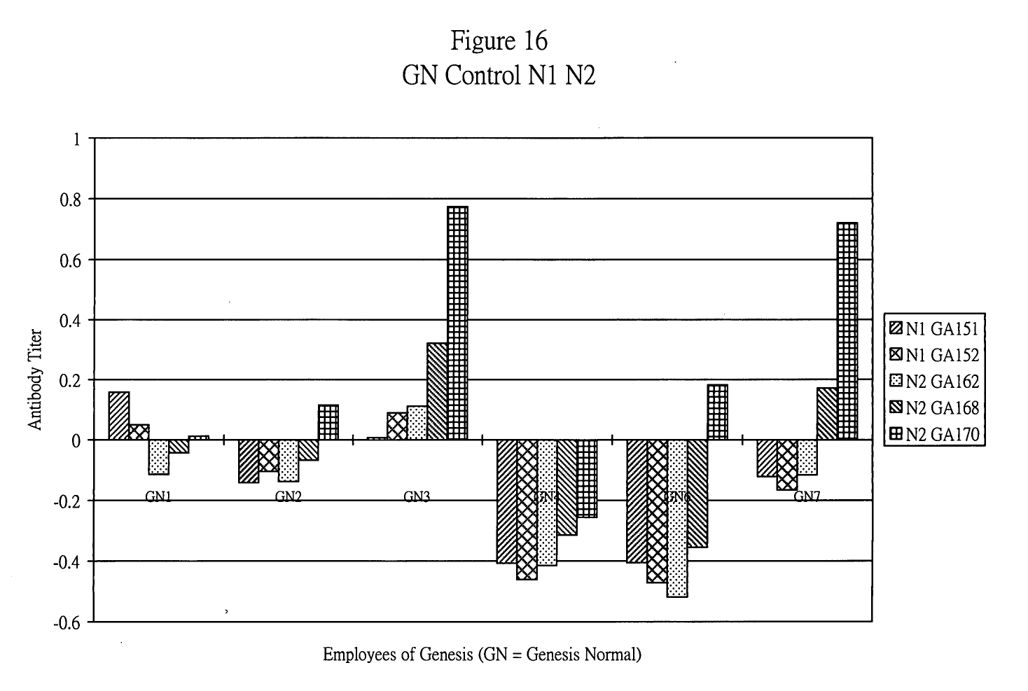

FIG. 17 is the epitope profiles of time course of SARS probable patient #51's sera IgG immune response in view of epitopes N1: GA51, GA152 and N2: GA162, GA168, GA170.

FIG. 18 is the epitope profiles of time course of SARS probable patient #50's sera IgG immune response in view of epitopes N1: GA151, GA152 and N2: GA162, GA168, GA170.

FIG. 19 is the epitope profiles of time course of SARS probable patient #47's sera IgG immune response in view of epitopes N1: GA151, GA152 and N2: GA162, GA168, GA170.

FIG. 20 is the epitope profiles of control subjects' sera IgG immune response in view of epitopes M: GA203, X2: GA230, GA231, and S2: GA287, GA291.

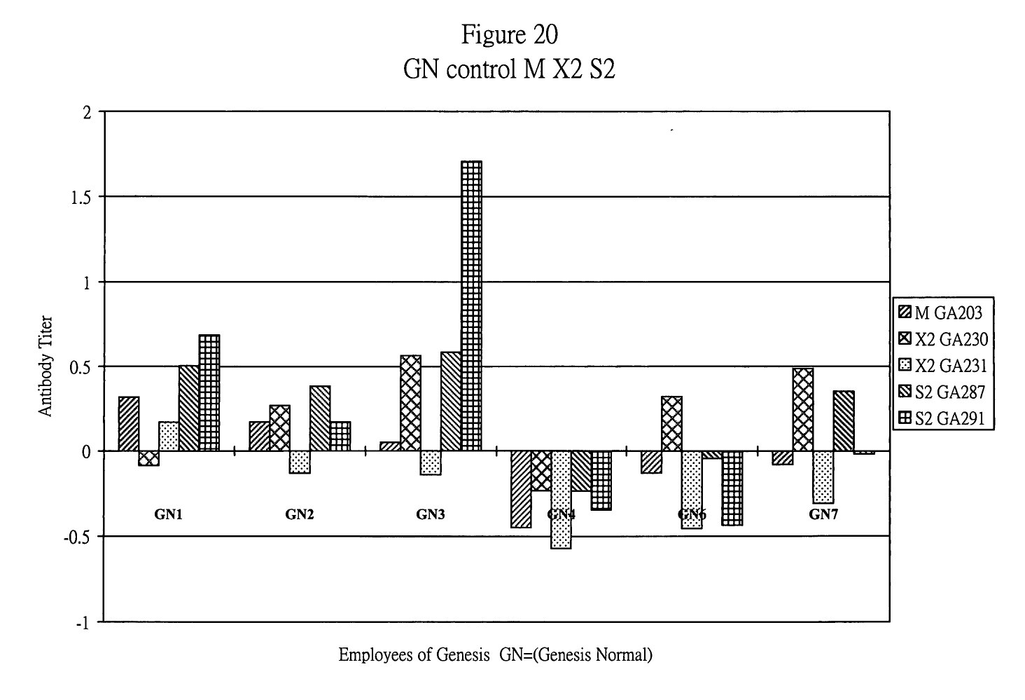

FIG. 21 is the epitope profiles of time course of SARS probable patient #51's sera IgG immune response in view of epitopes M: GA203, X2: GA230, GA231, and S2: GA287, GA291.

FIG. 22 is the epitope profiles of time course of SARS probable patient #50's sera IgG immune response in view of epitopes M: GA203, X2: GA230, GA231, and S2: GA287, GA291.

FIG. 23 is the epitope profiles of time course of SARS probable patient #47's sera IgG immune response in view of epitopes M: GA203, X2: GA230, GA231, and S2: GA287.

FIG. 24 is the epitope profiles of cross reactions between control polypeptides and chicken anti-avian infectious bronchitis virus (IBV) sera.

FIG. 25 is the epitope profiles of cross reactions between S1 polypeptides and chicken anti-avian infectious bronchitis virus (IBV) sera.

FIG. 26 is the epitope profiles of cross reactions between S2 polypeptides and chicken anti-avian infectious bronchitis virus (IBV) sera.

FIG. 27 is the epitope profiles of cross reactions between N1 polypeptides and chicken anti-avian infectious bronchitis virus (IBV) sera.

FIG. 28 is the epitope profiles of cross reactions between N2 polypeptides and chicken anti-avian infectious bronchitis virus (IBV) sera.

FIG. 29 is the epitope profiles of cross reactions between M polypeptides and chicken anti-avian infectious bronchitis virus (IBV) sera.

FIG. 30 is the epitope profiles of cross reactions between sE polypeptides and chicken anti-avian infectious bronchitis virus (IBV) sera.

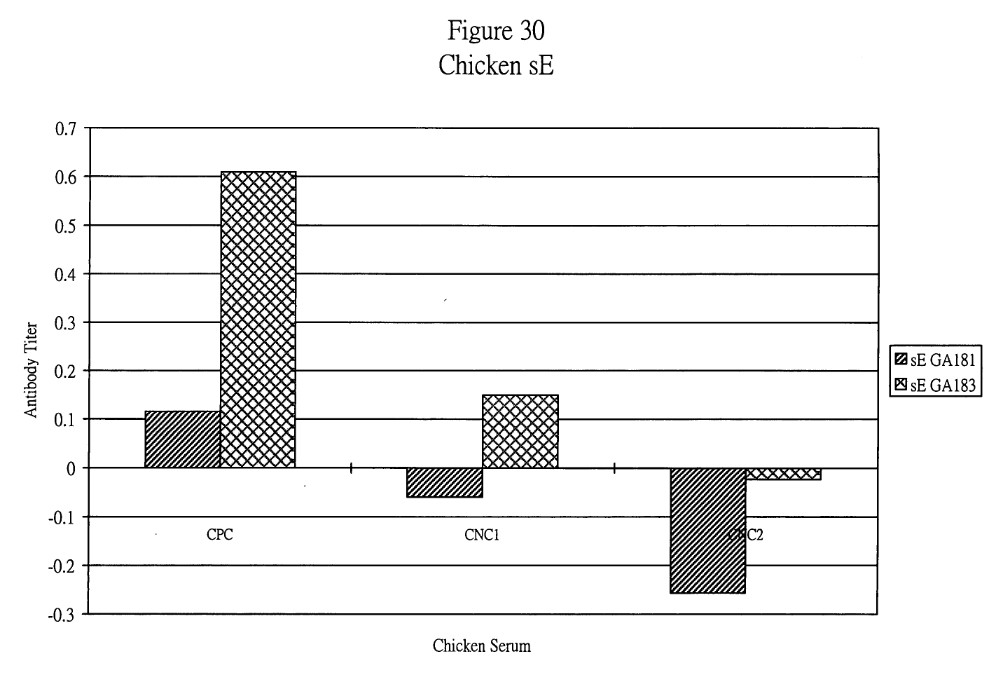

FIG. 31 is the epitope profiles of cross reactions between X1 polypeptides and chicken anti-avian infectious bronchitis virus (IBV) sera.

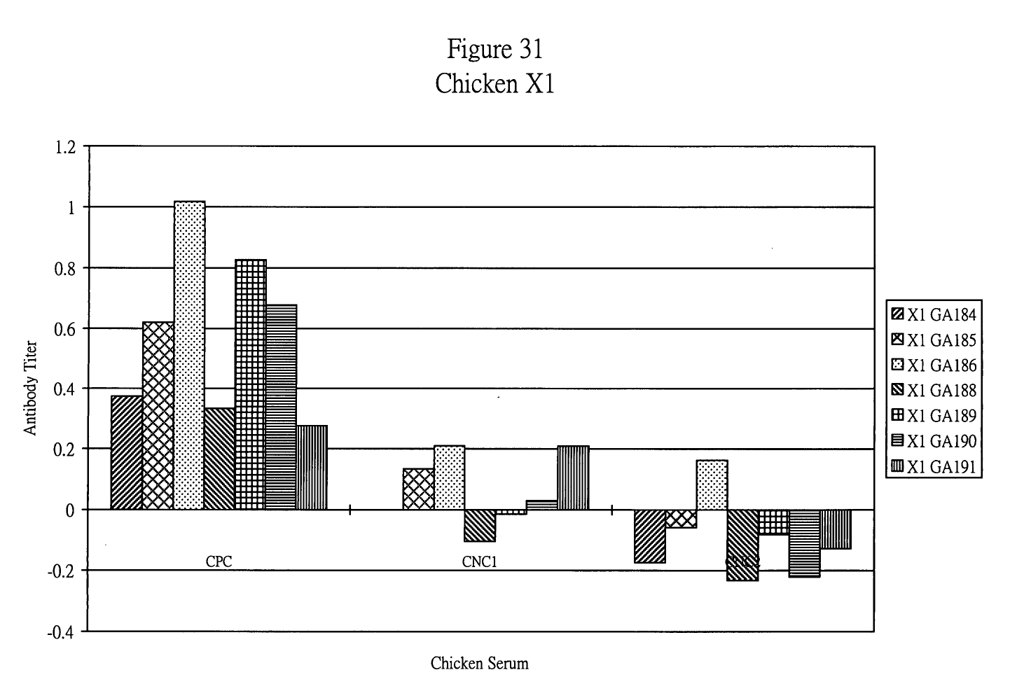

FIG. 32 is the epitope profiles of cross reactions between control polypeptides and cat anti-feline coronavirus sera.

FIG. 33 is the epitope profiles of cross reactions between S1 polypeptides and cat anti-feline coronavirus sera.

FIG. 34 is the epitope profiles of cross reactions between S2 polypeptides and cat anti-feline coronavirus sera.

FIG. 35 is the epitope profiles of cross reactions between N1 polypeptides and cat anti-feline coronavirus sera.

FIG. 36 is the epitope profiles of cross reactions between N2 polypeptides and cat anti-feline coronavirus sera.

FIG. 37 is the epitope profiles of cross reactions between M polypeptides and cat anti-feline coronavirus sera.

FIG. 38 is the epitope profiles of cross reactions between sE polypeptides and cat anti-feline coronavirus sera.

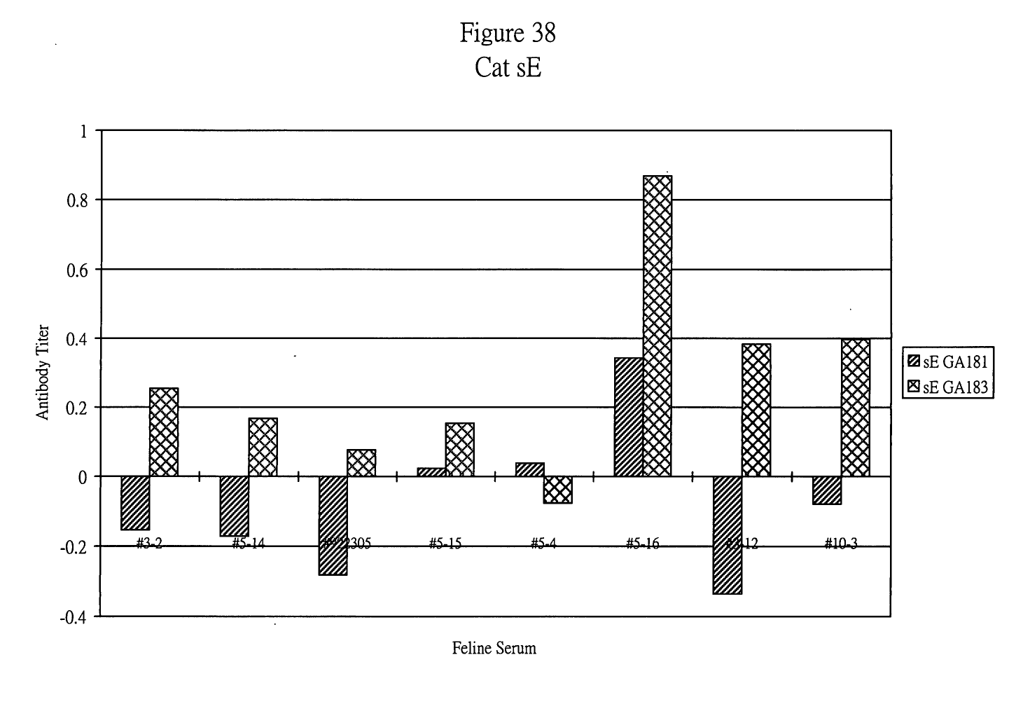

FIG. 39 is the epitope profiles of cross reactions between X1 polypeptides and cat anti-feline coronavirus sera.

FIG. 40 shows the immune response of serum from ducks immunized with S1 epitopes.

FIG. 41 shows the immune response of serum from ducks immunized with S2 epitopes.

FIG. 42 shows the immune response of serum from ducks immunized with N1 epitopes.

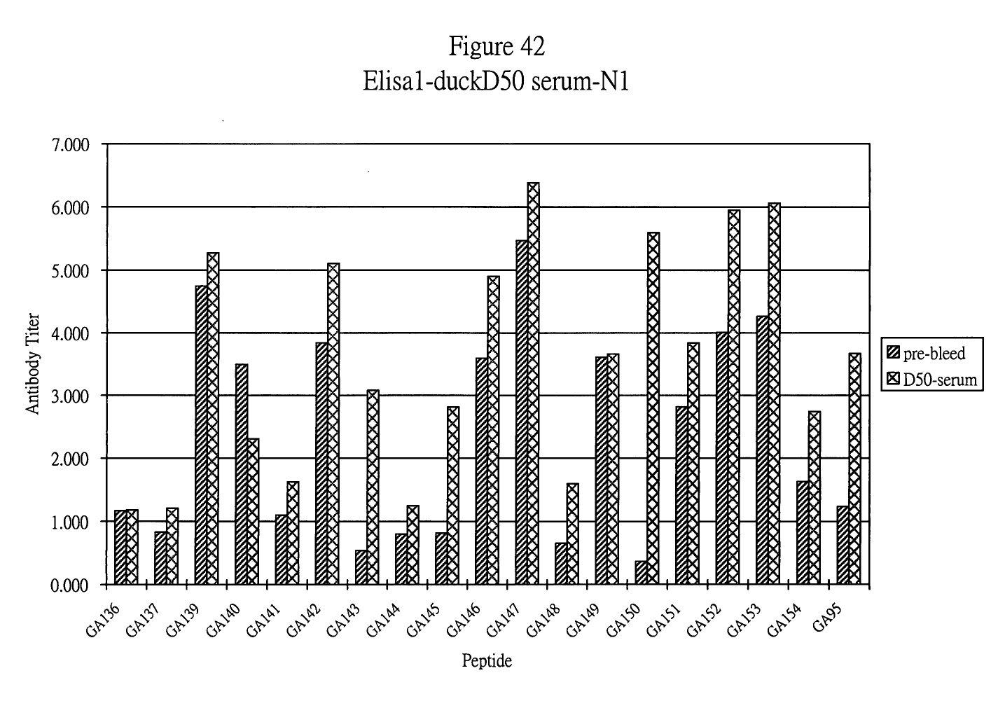

FIG. 43 shows the immune response of serum from ducks immunized with N2 epitopes.

FIG. 44 shows the immune response of serum from ducks immunized with M epitopes.

FIG. 45 shows the immune response of serum from ducks immunized with sE epitopes.

FIG. 46 shows the immune response of serum from ducks immunized with X1 epitopes.

FIG. 47 shows epitope profiles of time course of SARS probable patient #51's serum IgM response in view of N1: GA151, GA152, GA153, and GA154. The #51 patients' serum is diluted 300×.

Table 1 lists the zone, SEQ ID NO., peptide, sequence, location, annotation, format and notes of the synthetic SARS-COV related polypeptides.

### DESCRIPTION OF THE EMBODIMENTS

Definitions

The term “degenerate variant” as used herein refers to a polypeptide that has the same function but with one or more different amino acid(s) from mutation, substitution, addition or deletion or that is at least 90% identical to the original amino acid sequence.

The term “immunogenic composition” as used herein refers to a composition that provokes an immune response.

The term “immune response” as used herein refers to bodily response to an antigen that occurs when lymphocytes identify the antigenic molecule as foreign and induce the formation of antibodies and lymphocytes capable of reacting with it and rendering it harmless.

The term “linear form” as used herein refers to a single chain of amino acids.

The term “branched form” as used herein refers to the Multiple Antigenic Peptides (MAP), having at least two branches; it can be four, eight or more branches that result in a molecule which has a high molar ratio of peptide antigen to core molecule and, typically, will elicit a stronger anti-peptide antibody response.

SARS-COV Specific Epitopes

The present invention relates to a collection of one or more polypeptides from the 189 SARS-COV related polypeptides that permit an analysis of the epitope profiles of SARS-COV infected and non-infected human and animal sera. The collection is set forth in Table 1. The SARS-CoV specific epitopes were identified by comparison of each peptide's antibody binding activity in parallel in 5 human groups' sera (group 0 being Genesis' employees, group 1 being hospital employees, group 2 being SARS suspected patients, group 3 being SARS probable patients, and group 4 being recovering patients from groups 2 and 3). The specific epitopes identified are further categorized into two groups, i.e. the most specific epitopes and the less specific epitopes, also referred to as the second specific epitopes.

The most specific epitopes are the SARS-CoV nucleocapsid protein N1: GA137 (SEQ ID NO: 46), GA139 (SEQ ID NO: 47), GA142 (SEQ ID NO: 50), GA146 (SEQ ID NO: 54), GA147 (SEQ ID NO: 55), GA151˜GA153 (SEQ ID NO: 59-SEQ ID NO: 61); N2: GA156 (SEQ ID NO: 65), GA 160 (SEQ ID NO: 69), GA164 (SEQ ID NO: 73), GA166 (SEQ ID NO: 75), GA 167 (SEQ ID NO: 76), GA169 (SEQ ID NO: 78), GA170 (SEQ ID NO: 79); and protein X2: GA231 (SEQ ID NO: 140), which polypeptides bound with SARS probable patients' serum (group 3) more strongly (usually AT>2.5) than other non-SARS sera (group 0 and group 1).

The second specific epitopes are the SARS-COV nucleocapsid protein N1: GA149 (SEQ ID NO: 57), GA150 (SEQ ID NO: 58), GA154 (SEQ ID NO: 62); N2: GA161 (SEQ ID NO: 70), GA162 (SEQ ID NO: 71), GA165 (SEQ ID NO: 74), GA 168 (SEQ ID NO: 77); matrix protein (M): GA203 (SEQ ID NO: 112); spike protein (S): GA132 (SEQ ID NO: 40), GA134 (SEQ ID NO: 42), GA287 (SEQ ID NO: 183), GA291 (SEQ ID NO: 187); protein X2: GA230 (SEQ ID NO: 139); and protein X4: GA247 (SEQ ID NO: 156), which polypeptides bound with SARS probable patients' serum (group 3) more strongly (1<AT<2.5) than the other non-SARS sera (group 0, group 1).

An analysis, such as a serum ELISA, using an epitope profile containing SARS-COV S1, S2, N1, N1, M, sE, X1, X2, X3, X4, and X5 proteins (SEQ ID NO: 7-SEQ ID NO: 195) can provide information on the status of SARS-CoV infection. Also useful is the epitope profiles of N1 proteins (SEQ ID NO: 44-SEQ ID NO: 62), N2 proteins (SEQ ID NO: 63-SEQ ID NO: 82), or X2 proteins (SEQ ID NO: 124-SEQ ID NO: 142) containing the most specific epitopes and epitope profiles of M proteins (SEQ ID NO: 83-SEQ ID NO: 89 and SEQ ID NO: 112-SEQ ID NO: 123) and S proteins (SEQ ID NO: 7-SEQ ID NO: 43) containing the second specific epitopes.

Specifically, using the most specific epitope profile of the GA151, GA152, and GA170 polypeptides, one can differentiate the 23 SARS suspected patients (group 2) to be real SARS-COV infected (17 cases) or SARS-COV non-infected cases (6 cases) (data set forth below). Moreover, by using the most specific epitopes profile ELISA method, the anti-SARS-CoV immune response can be detected two days earlier than the RT-PCR method of virus RNA detection (data set forth below).

Moreover, among the specific epitopes, certain epitopes have immediate early antibody binding activities (days 1-6) and early antibody binding activities (days 7-29). These were identified in parallel analysis of the epitope profiles of serum taken from different time points. The SARS-COV infected patients' serum bound to the following immediate early epitopes: X2: GA230 (SEQ ID NO: 139), GA231 (SEQ ID NO: 140); S2: GA287 (SEQ ID NO: 183); M: GA203 (SEQ ID NO: 112); N1: GA151 (SEQ ID NO: 59), GA152 (SEQ ID NO: 60); N2: GA168 (SEQ ID NO:77), GA170 (SEQ ID NO: 79); and S2: GA291 (SEQ ID NO:187), and to the following early epitope: N2: GA162 (SEQ ID NO: 71).

Each of these epitopes and the epitope profiles have research, diagnostic, and therapeutic values.

SARS-COV Non-Specific Epitopes

The invention also describes SARS-COV epitopes that are not specific to SARS-CoV antibody containing sera. These non-specific epitopes have antibody binding activities in all five groups of human sera, and they are S1: GA 101 (SEQ ID NO: 11), GA102 (SEQ ID NO: 11), GA102 (SEQ ID NO: 12), S2: GA117 (SEQ ID NO: 25), N2: GA155 (SEQ ID NO: 64), GA158 (SEQ ID NO: 67), GA172 (SEQ ID NO: 81), M: GA179 (SEQ ID NO: 88), GA209 (SEQ ID NO: 118). These non-specific epitopes may be used in affinity purification and or preabsorption of antisera as is routinely performed by one skilled in the art.

SARS-CoV Inflammation Epitopes

Some inflammation epitopes have been identified. These represent polypeptides that frequently bound with SARS probable patients' serum more strongly during the treatment period than during the recovery period after discharge from hospital. They include both SARS-COV specific and non-specific epitopes, i.e. SARS-CoV spike protein: GA01 (SEQ ID NO: 11), GA102 (SEQ ID NO: 12), GA117 (SEQ ID NO: 25), GA132 (SEQ ID NO: 40), GA134 (SEQ ID NO: 42), nucleocapsid protein: GA137 (SEQ ID NO: 46), GA139 (SEQ ID NO: 49), GA142 (SEQ ID NO: 50), GA143 (SEQ ID NO: 51), GA146 (SEQ ID NO: 54), GA147 (SEQ ID NO: 55), GA149˜GA154 (SEQ ID NO: 57-SEQ ID NO: 62), GA155 (SEQ ID NO: 64), GA156 (SEQ ID NO: 65), GA158 (SEQ ID NO: 67), GA160-GA162 (SEQ ID NO: 69-SEQ ID NO: 71), GA164˜GA170 (SEQ ID NO: 73-SEQ ID NO: 79), matrix protein: GA179 (SEQ ID NO: 88) and protein X2: GA231 (SEQ ID NO: 140).

The use of inflammation epitopes that show strong SARS sera antibody binding activities only during the treatment phase in the hospital can, by subtraction, lead to the identification of SARS-CoV specific epitopes that bind strongly even after hospitalization, i.e. subtracting SARS-CoV specific inflammation epitopes from the total SARS-COV specific epitopes. The identification of SARS-CoV specific epitopes that bind strongly even after hospitalization allow identification of people who were infected with SARS-COV but are in the recovery period.

Immunogenic Peptides

Certain polypeptides can be used as immunogens (immunogenic peptides) to raise corresponding antibody in hosts such as mice, rabbits, and ducks. There is a high immune response when compared with pre-immunization (pre-bleed) serum when using the following peptide immunogens: S1: GA100˜GA102 (SEQ ID NO: 10-SEQ ID NO: 12), GA91 (SEQ ID NO: 7), GA109 (SEQ ID NO: 17), GA111 (SEQ ID NO: 19), GA113 (SEQ ID NO: 21), S2: GA117 (SEQ ID NO: 25), GA119 (SEQ ID NO: 27), GA128˜GA130 (SEQ ID NO: 36-SEQ ID NO: 38), N1: GA143 (SEQ ID NO: 51), GA145 (SEQ ID NO: 53), GA95 (SEQ ID NO:44), GA150˜GA154 (SEQ ID NO: 58-SEQ ID NO: 62), N2: GA155 (SEQ ID NO:64), GA156 (SEQ ID NO: 65), GA158˜GA161 (SEQ ID NO: 67-SEQ ID NO: 70), GA96 (SEQ ID NO: 63), GA167 (SEQ ID NO: 76), GA173 (SEQ ID NO: 82), M: GA174 (SEQ ID NO: 84), GA93 (SEQ ID NO: 83), sE: GA181 (SEQ ID NO: 90), and protein X1: GA186 (SEQ ID NO: 95), GA188˜GA190 (SEQ ID NO:97-SEQ ID NO: 99).

The availability of SARS-CoV epitope sequences profiles should be useful in controlling the disease by making it possible to develop diagnostic tests, vaccines, and antiviral agents.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention. Further, all publications mentioned herein are incorporated by reference.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

It must be noted that, as used herein and in the appended claims, the singular forms “a,” “or,” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

The following examples further illustrate the invention. They are merely illustrative of the invention and disclose various beneficial properties of certain embodiments of the invention. The following examples should not be construed as limiting the invention.

### EXAMPLES

The following examples illustrate the identification, design and production of SARS-COV epitope related synthetic polypeptides and production of polyclonal antibodies using these polypeptides.

### Example 1

### Peptide Synthesis

Polypeptides were synthesized by using solid phase peptide synthesis strategy. The standard F-moc chemistry was performed on an Advanced ChemTech's Peptide Synthesizer, Model Apex 396, according to manufacturer's instructions. The branched multiple antigenic peptides (GA98˜GA191, GA283˜GA298) were synthesized from the K_{core} wang resin (heptalysyl core K₄K₂K) which was purchased from Novabiochem. For linear polypeptide synthesis (GA91˜GA96 and GA192˜GA269), we used wang resin coupled with the first amino acid of the C-terminal as solid support. After complete synthesis of the peptide, the resin was treated with cleavage cocktails [TFA(trifluoroacetic acid), TIS(triisipropyl silane), EDT(ethanol dithiol)] according to standard procedures used to cleave the peptide from the resin and deprotect the protecting groups on the amino acid side chains. For quality control of the polypeptides synthesis, each peptide was analyzed with HPLC and MALDI-TOF Mass spectrum methods.

### Example 2

### Peptide Epitope Annotation

Different polypeptides were synthesized based on the published genome sequence of SARS-CoV and were given the following annotations.

Six non-SARS-CoV polypeptides (GA6, GA53, GA64, GA81, GA83, GA84) were used as control polypeptides.

The synthetic polypeptides of SARS-COV spike (S) protein included amino acids (aa) sequence 421-520 (GA91, GA98˜GA116), within the S1 region. Also included were aa 1021˜1120 (GA117˜GA135) and aa 1116˜1200 (GA283˜GA298), both of which were within the S2 region.

The synthetic polypeptides of SARS-CoV nucleocapsid (N) protein included aa 70-169 (GA95, GA136˜GA154), defined as the N1 region and aa 329-350, 300-399 (GA 96, GA155˜GA173), defined as the N2 region.

The synthetic polypeptides of SARS-COV matrix membrane (M) protein included aa 1-20 (GA93, GA174˜GA176), aa 61˜85 (GA177˜GA180), aa 95˜124 (GA203˜GA207), and aa 164˜203 (GA208˜GA214) which were defined as M.

The synthetic polypeptides of SARS-COV small envelope (sE) protein included aa 1˜20 (GA181˜GA183) and aa 16˜76 (GA192˜GA202), which were defined as sE.

The synthetic polypeptides of SARS-COV protein X1 (X1) included aa 1˜40 (GA184˜GA190) and aa 93˜102 (GA191), which were defined as X1.

The synthetic polypeptides of SARS-COV protein X2 (X2) included aa 24-98, 119˜149 (GA215˜GA233), which were defined as X2.

The synthetic polypeptides of SARS-COV protein X3 (X3) included aa 163 (GA234˜GA244), defined as X3.

The synthetic polypeptides of SARS-COV protein X4 (X4) included aa 70-119 (GA245˜GA253), defined as X4.

The synthetic polypeptides of SARS-COV protein X5 (X5) included aa 1-84 (GA254˜GA269), defined as X5.

Genes encoding protein X1 and X2 are found within the small open reading frames (ORFs) between the S and sE genes. Genes encoding proteins X3, X4, and X5 are found within the ORFs between the M and N genes.

A table of the synthetic polypeptides is shown in Table 1.

### Example 3

### ELISA

The enzyme linked immunosorbent assay (ELISA) was used to screen for the existence of anti-SARS-CoV antibody in human and animal sera. The ELISA was conducted by coating polystyrene 96 well plates with 1 μg/well of polypeptides. For comparison of results from each group in parallel, experiments were done at the same time. The antibody titer (AT) was defined as (O.D.₄₀₅ value of Target —O.D.₄₀₅ value of Blank)/O.D.₄₀₅ value of Blank) to reduce the variation of different time performance to the least extent. The primary sera were all diluted 3000× in 1% BSA/PBS, and the secondary antibody was HRP labeled goat anti-human IgG(H+L) (Pierce Biotechnology Inc.) diluted 5000× or Donkey anti-chicken IgY (IgG) (H+L) (Research Diagnostics, Inc.) diluted 5000× or goat anti-cat IgG (H+L) (Research Diagnostics, Inc.) diluted 5000×. Using TMB (3,3′, 5, 5′ tetramethyl benzidine) as the substrate, generally, the immune response was defined as elevated significantly when the antibody titer (AT) was more than 1 in human serum and more than 0.8 in animal serum.

### Example 4

### Grouping of Human and Animal Sera Samples

Human Sera

There were 74 human sera samples divided into five groups. The groups were group 0: the sera of five to seven normal humans sampled from employees of Genesis Biotech Inc. who didn't work in the hospital; group 1: the sera of eighteen humans who worked in the hospital and may have came in contact with SARS patients or related specimens (healthcare workers) (#1˜#18); group 2: the sera of twenty-three “SARS suspected patients”, identified according to the WHO case definition standard (#19˜#41); group 3: the sera of ten “SARS probable patients”, identified according to the WHO case definition standard (#42˜#51); group 4: the sera of eighteen humans, originally from group 2 and group 3 (#52˜#69), who had been discharged from the hospital and had recovered from the therapy of “SARS suspected” or “SARS probable”cases.

Blood samples were also collected from patients at different time points of the infection to examine putative time-dependent epitopes of SARS-CoV found in the infected patients. Three SARS probable patients' (#51, #50, and #47 patients) sera were collected at several time points.

Probable SARS patient #51: male. 35 years old

The patient record is as follows:

May 8, 2003—hospitalization
5/9—chest radiograph shows interstitial infiltrate
5/10—fever, diarrhea twice
5/11-fever
5/13—RT-PCR diagnosis of SARS-COV was positive, SD rapid test
(Standard Diagnostics Inc.) of SARS-COV antibody was negative
5/15—fever
5/16—body temperature back to normal
5/18—chest radiograph shows ok
6/5—clinical syndrome recovered
6/12—bleed serum after recovery
   Blood samples from patient #51 were collected at various time points as follows:
#51A: day 0, May 11, 2003, fever, SD rapid test of SARS-COV antibody was negative
#51B: day 2E (“early”, in the mornings), May 13, 2003, SD rapid test of SARS-CoV antibody was negative
#51C: day 2L (“late”, in the afternoons), May 13, 2003, RT-PCR diagnosis of SARS-CoV was positive, SD rapid test of SARS-COV antibody was negative
#51D: day 4, May 15, 2003, fever, SD rapid test of SARS-COV antibody was negative
#51E: day 5E, May 16, 2003, body temperature back to normal, SD rapid test of SARS-CoV antibody Was negative
#51F: day 5L, May 16, 2003, SD rapid test of SARS-COV antibody was negative
#51G: day 8, May 19, 2003, SD rapid test of SARS-COV antibody was positive
#51H: day 12, May 23, 2003, SD rapid test of SARS-COV antibody was positive
#51K: day 25, Jun. 5, 2003, clinical syndrome recovered, SD rapid test of SARS-CoV antibody was positive
#51L: day 32, Jun. 12, 2003, discharged from hospital, bleed serum after recovery
#51M: day 41, Jun. 21, 2003, bleed serum after recovery
   Probable SARS patient #50: female, 58 years old
   Patient record is as follows:
   May 3, 2003—transferred from another hospital, had fever prior to transfer, hospitalization, body temperature was normal
   5/6—chest radiograph showed recovering
   5/13—bleed serum during hospitalization, RT-PCR diagnosis negative of SARS-CoV, SD rapid test of SARS-CoV antibody positive
   5/15—body temperature normal
   5/20—bleed serum after recovery
   5/25—body temperature normal
      Blood samples from patient #50 were collected at various time points as follows:
#50A: day 0, May 10, 2003, SD rapid test of SARS-CoV antibody was positive
#50B: day 2, May 12, 2003, SD rapid test of SARS-COV antibody was positive
#50C: day 3, May 13, 2003, bleed serum during hospitalization
#50D: day 10, May 20, 2003, discharged from hospital, bleed serum after recovery
#50E: day 16, May 26, 2003, bleed serum after recovery
#50F: day 27, Jun. 6, 2003, bleed serum after recovery
#50G: day 55, Jul. 4, 2003, bleed serum after recovery
   Probable SARS patient #47: male, 35 years old
   The patient record is as follows:
   May 22, 2003—hospitalization
   5/23—fever
   5/24—fever, SD rapid test of SARS-COV antibody was positive
   5/25—body temperature was normal, chest radiograph showed recovering
      Blood samples from patient #47 were collected at various time points as follows:
#47A: day 0, May 26, 2003, SD rapid test of SARS-CoV antibody was positive
#47B: day 2, May 28, 2003, bleed serum during hospitalization, RT-PCR of SARS-COV was negative, SD rapid test of SARS-COV antibody was positive
#47C: day 7, Jun. 2, 2003, discharged from hospital, bleed serum after recovery
#47D: day 22, Jun. 17, 2003, discharged from hospital, bleed serum after recovery
#47E: day 28, Jun. 23, 2003, discharged from hospital, SD rapid test of SARS-CoV antibody was positive
   Animal Sera

The cross reaction of animal coronavirus (infectious bronchitis virus and feline coronavirus antibody positive and negative sera with the SARS-CoV peptide epitopes were examined in 3 chicken samples and 8 cat samples. The three chicken samples were one chicken positive control (CPC) of anti-avian infectious bronchitis virus (IBV) and two chicken negative controls (CNC 1 and CNC 2) using specific pathogen free (SPF) chicken sera. The eight cat samples include four sera samples from 4 cats with feline coronavirus (FCV) diagnosis-RT-PCR positive and antibody rapid test positive: #5-4, #5-16, #3-12, #10-3; one sample from 1 cat with FCV-RT-PCR negative and antibody rapid test positive: #5-15; one sample from 1 cat with FCV-RT-PCR positive and antibody rapid test negative: #5-14; one sample from 1 cat with FCV-RT-PCR positive and antibody rapid test not performed: #922305; one sample from 1 cat with FCV-RT-PCR negative and antibody rapid test negative: #3-2. These sera were analyzed by standard ELISA method.

### Example 5

### Identification of SARS Related Epitopes

To identify potential epitopes that would be recognized by the SARS-CoV antibodies, sera samples from Groups 0-4 were analyzed with ELISA plates coated with synthetic SARS-COV polypeptides spanning the regions of S, N, M, sE, X1, X2, X3, X4, and X5 and control polypeptides.

Peptide array was used to detect the potential epitope binding activity of human sera of normal and infected subjects. The peptide array covered the sequential peptide sequences with 4˜6 amino acids overlapping in adjacent wells. These branched or linear polypeptides included the following SARS-CoV proteins:
spike glycoprotein (S): S1: 437th aa˜461th aa (GA91), aa 421-520 (GA98˜GA116), S2: 1021˜1120 (GA117˜GA135), 1116˜1200 (GA283˜GA298)
nucleocapsid phosprotein (N): N1: aa 107˜126 (GA95), 70˜169 (GA136˜GA154) N2: aa 329-350, 300˜399 (GA 96, GA155˜GA173)
matrix protein (M): aa 1˜20 (GA93, GA174˜176), 61 ˜85 (GA177˜GA180), 95˜124 (GA203˜GA207), 164˜203 (GA208˜GA214)
small envelope protein (sE): aa 1˜20 (GA181˜GA183), 16˜76 (GA192˜GA202)
protein X1 (X1): aa 1˜40 (GA184˜GA190), 93˜102 (GA191)
protein X2 (X2): aa 24˜98 (GA215˜GA228), 119˜149 (GA229˜GA233)
protein X3 (X3): aa 1˜63 (GA234˜GA244)
protein X4 (X4): aa 70˜119 (GA245˜GA53)
protein X5 (X5): aa 1˜84 (GA254˜GA269)
control polypeptides: GA6 (28 kDa structural protein of VP28 of shrimp white spot syndrome virus (WSSV) 52th aa ˜66th aa), GA53 (hemagglutinin of Influenza A virus (H3N2) 306th aa ˜313th aa fused with VP1 of enterovirus 714th aa ˜10th aa), GA64 (coat protein of fish nervous necrosis virus (NNV) 274th aa ˜289th aa), GA81 (spike glycoprotein of rabies virus 355th aa ˜364th aa fused with cyclic 157th aa ˜172th aa of peptide sequence of latent membrane protein LMP-1 of human herpesvirus 4/Epstein-Barr Virus), GA83 (4th aa ˜30th aa of membrane matrix protein M1 of Influenza A virus), and GA84 (25th aa ˜41th aa of membrane matrix protein M1 of Influenza A virus).

The B cells immune response (antibody) to multiple SARS-COV synthetic polypeptides (S, N, M, sE, X1, X2, X3, X4, and X5) and control polypeptides were analyzed with peptide array-ELISA and serum from group 0 to group 4 in parallel. When the antibody titer index (AT) was more than 1, it suggested significant epitope (or peptide) binding activity of the serum examined.

SARS-COV Specific Epitopes

In the peptide array-serum interaction, the SARS specifically related epitopes, also referred to as SARS-CoV specific epitopes, were defined by having both a positive signal in SARS-probable and or SARS-suspected cases and a negative signal in the other non-SARS-CoV infected groups (group 0 and group 1). Generally, AT>1 is positive, AT<1 is negative; however, for some epitopes a value of AT<2 was designated as a negative response. Many SARS-COV specific eiptopes were identified and they were further categorized as the most specific epitopes or the second specific epitopes according to their anti-SARS-CoV antibody binding activities in different groups.

Most Specific

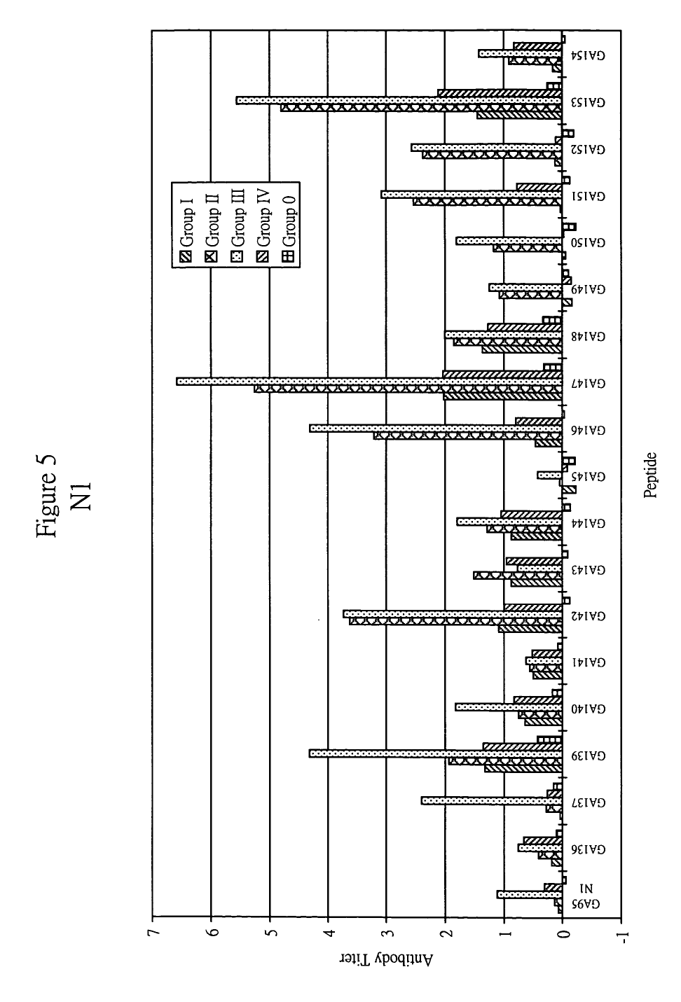

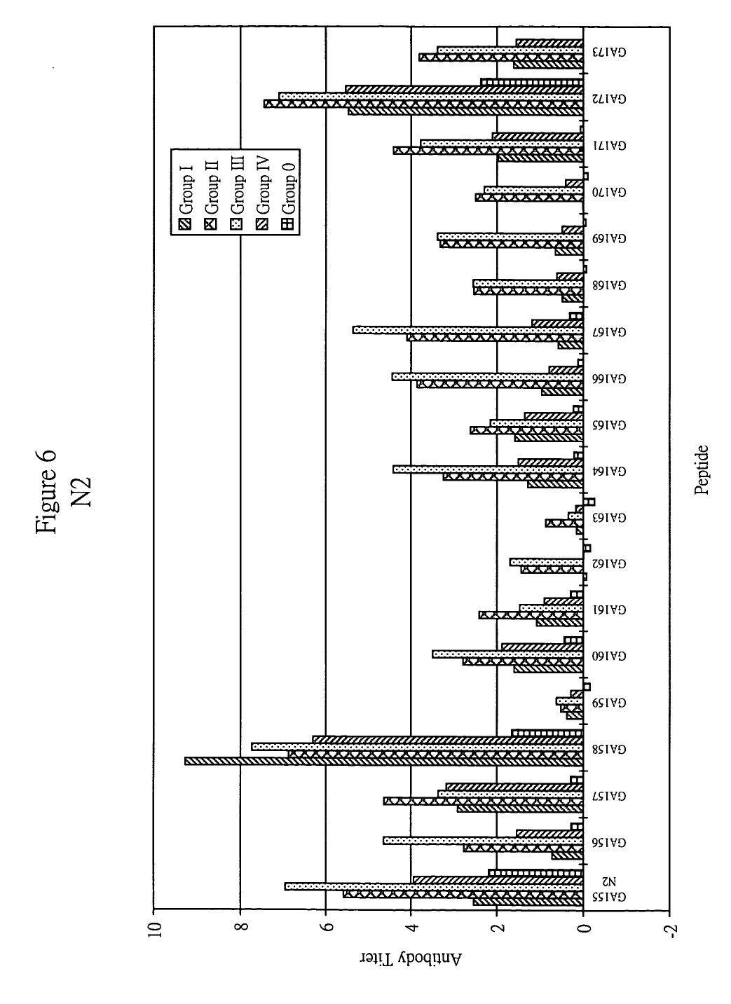

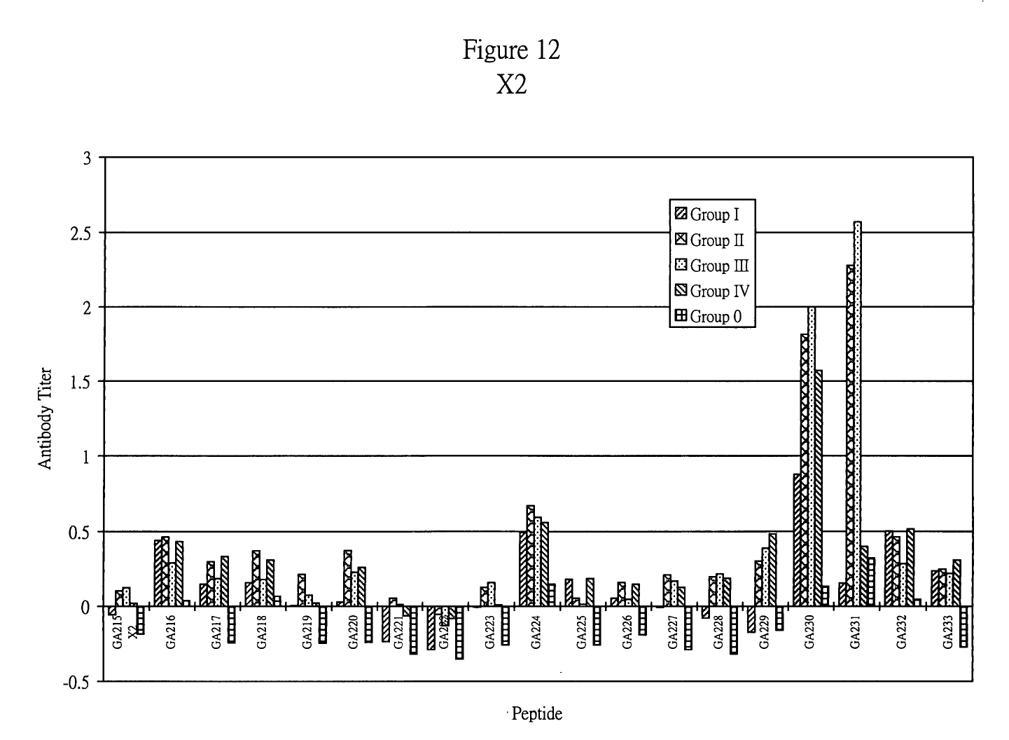

The most specific epitopes reacted with sera of SARS-probable patients (group 3) more strongly (usually AT>2.5) than with other non-SARS sera. They were: SARS-COV nucleocapsid protein (N): aa sequence N1: 75-94 (GA137, GA139), 100-109 (GA142), 120-134 (GA146, GA147), 145-164 (GA151GA153), N2: 305-314 (GA156), 325-334 (GA160), 345-354 (GA164), 355-364 (GA166), 360-369 (GA167), 370-379 (GA169), 375-384 (GA170) and protein X2: 129-138 (GA231) (see FIGS. 5, 6, and 12).

Second Specific Epitopes

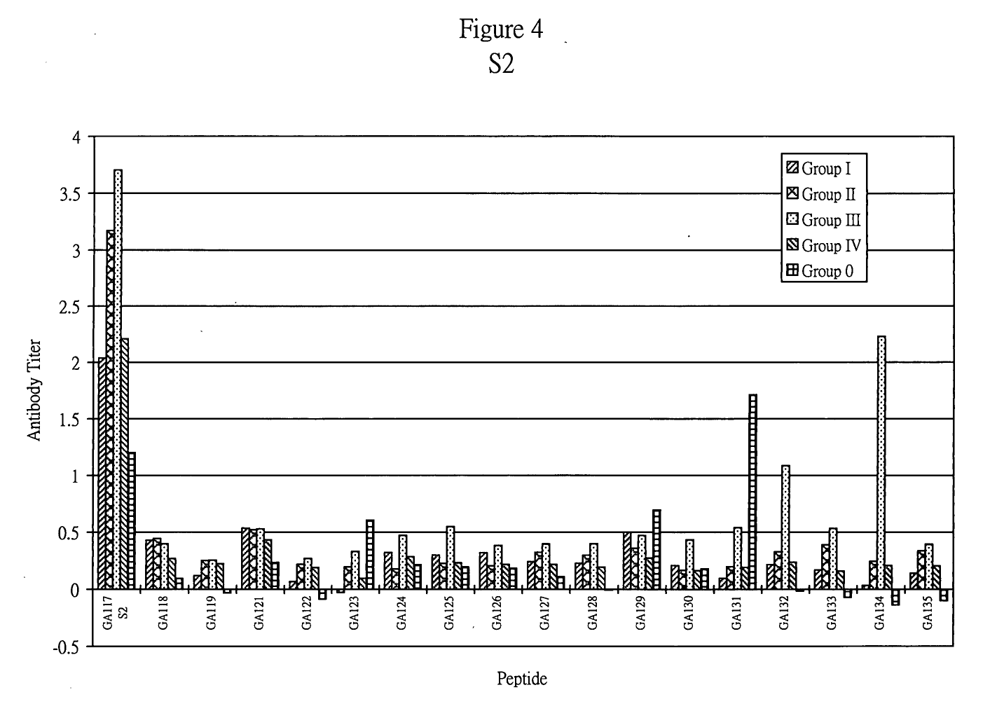

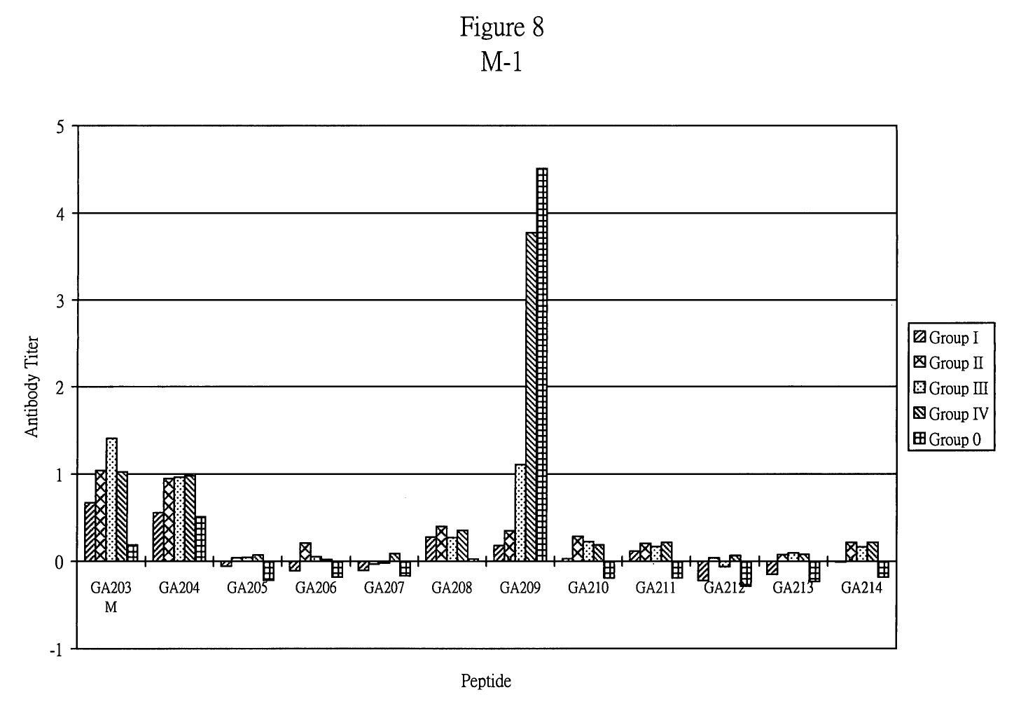

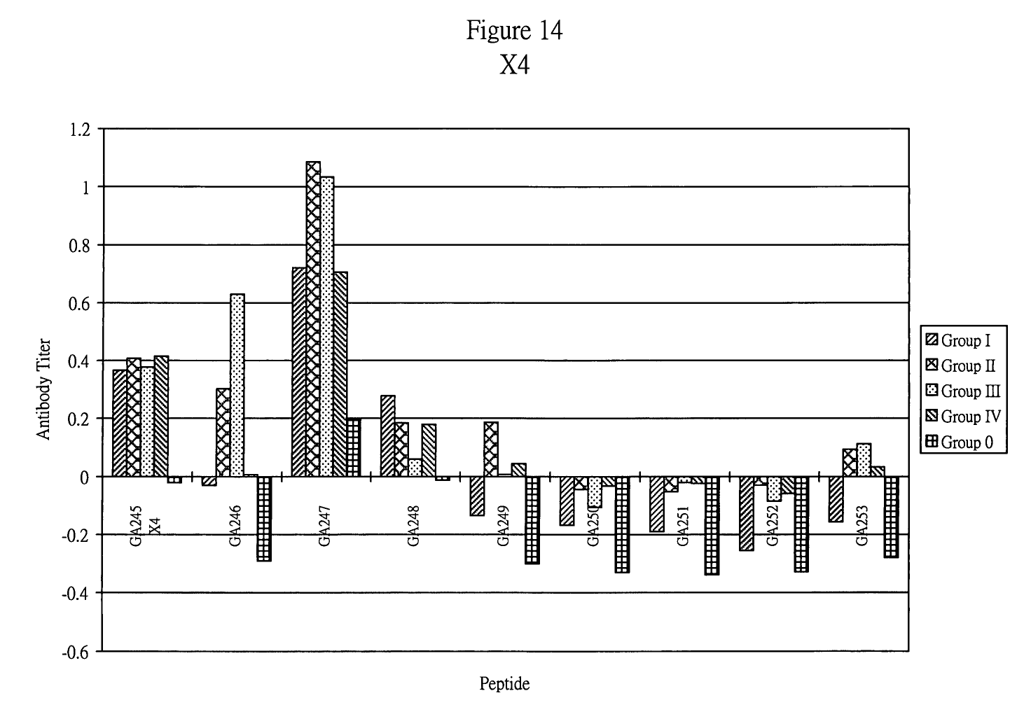

The second specific epitopes were the nucleocapsid protein N1: GA149, GA150, GA154; N2: GA161, GA162, GA165, GA168; matrix protein (M): GA203; spike protein (S): GA132, GA134, GA287, GA291; protein X2: GA230; and protein X4: GA247, which polypeptides bound with SARS probable patients' serum (group 3) more strongly (1<AT<2.5) than the other non-SARS sera (group 0, group 1) (see FIGS. 4, 5, 6, 8, 12, and 14).

Using the most specific epitope profile containing GA151, GA152, GA170, one can differentiate the 23 SARS suspected patients (group 2, #19-#41) to be real SARS-COV infected (17 cases: #19, #21, #22, #23, #24, #25, #26, #27, #28, #30, #31, #32, #36, #38 #39, #40, #41) or SARS-COV non-infected cases (6 cases: #20, #29, #33, #34, #35, #37). In other words, 74% of group 2 can be regarded as probable SARS patients, and 26% of group 2 can be regarded as non-probable SARS patients. The Responder (AT>1, positive)/Total samples (R/T) examined in group 3 (SARS probable patients, #42˜#51) was 10/10=100% when using the same epitope profile (GA151, GA152, GA170) as an anti-SARS-CoV antibody binding markers. In contrast, the R/T value=0 (0/18) in group 1 (normal human control of hospital employees) and group 0 (normal human control subjects outside of the hospital).

SARS-CoV Non-Specific Epitopes

Epitopes that had binding activities (AT>1) in all five groups of sera, sera from normal, infected, and recovering subjects, were designated as non-specific epitopes. These epitopes were GA101, GA102, GA117, GA155, GA158, GA172, GA179, and GA209.

Inflammation Epitopes

Certain immune response elevated (inflammation) epitopes were also identified (including both SARS-CoV specific and non-specific epitopes), which polypeptides frequently bound with SARS probable and suspected patients' serum more strongly during the treatment than during the recovery period after discharge from the hospital. They include SARS-COV spike protein: GA101, GA102, GA117, GA132, GA134, nucleocapsid protein: GA137, GA139, GA142, GA143, GA146, GA147, GA149˜GA154, GA155, GA156, GA158, GA160˜GA162, GA164˜GA170, matrix protein: GA179 and protein X2: GA231. The antibody titer for these polypeptides was higher during hospital therapy in contrast to the lower antibody titer in the recovery period after discharge from the hospital (patient #19 (hospitalization period) vs. #61 (post-hospitalization period), #22 vs. #62, #24 vs. #67, #25 vs. #70, #26 vs. #71, #28 vs. #60, #29 vs. #72, #30 vs. #63, #31 vs. #68, #32 vs. #65, #36 vs. #69, #37 vs. #52, #41 vs. #59, #42 vs. #73, #47 vs. #66, #50 vs. #58, #51 vs. #55).

In sum, ELISA analysis of SARS-infected human sera with SARS-COV polypeptides, certain epitopes were identified as SARS-specifically related epitopes, non-specific epitopes, and inflammation epitopes.

### Example 6

### Determination of Time-Dependent Epitope Profiles of SARS-COV

SARS-CoV specific epitopes' time dependency was investigated.

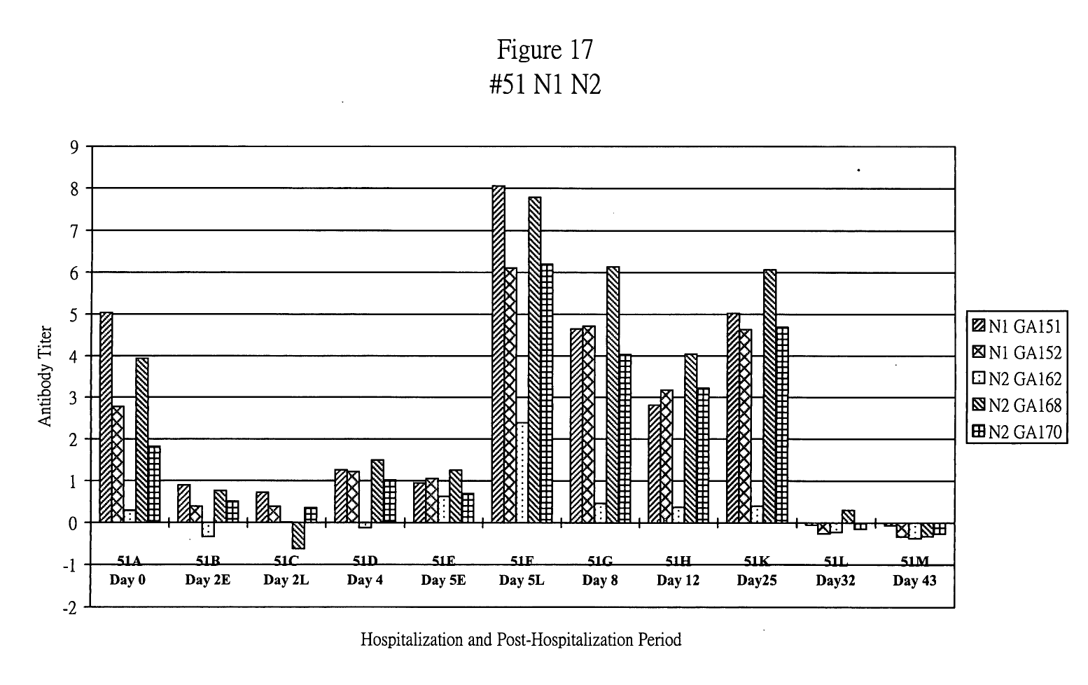

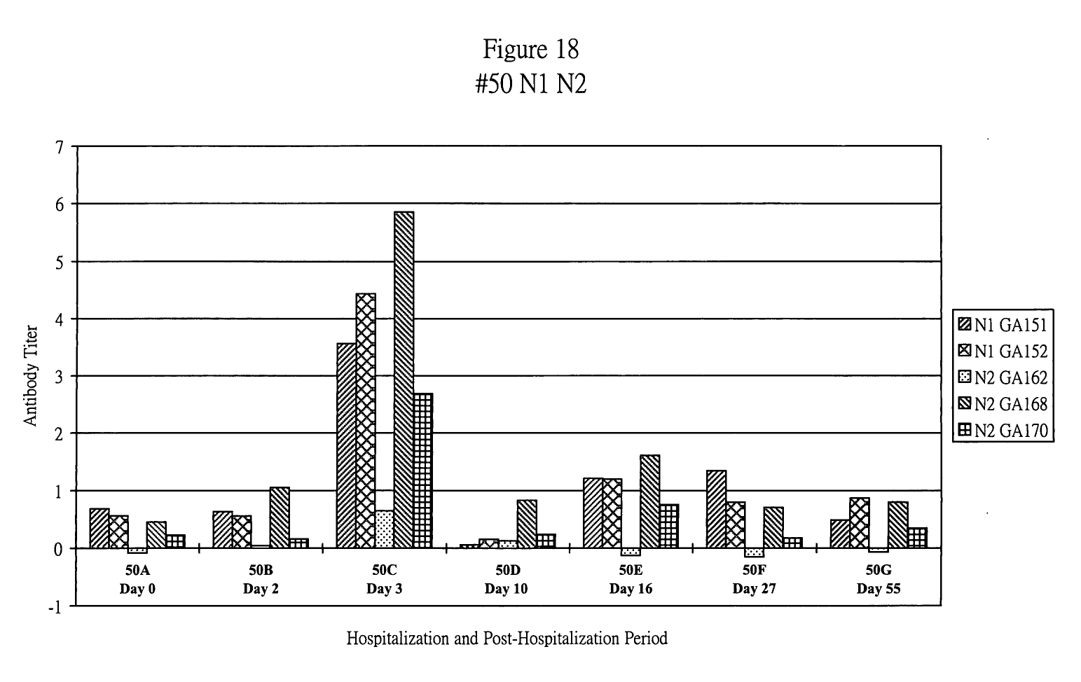

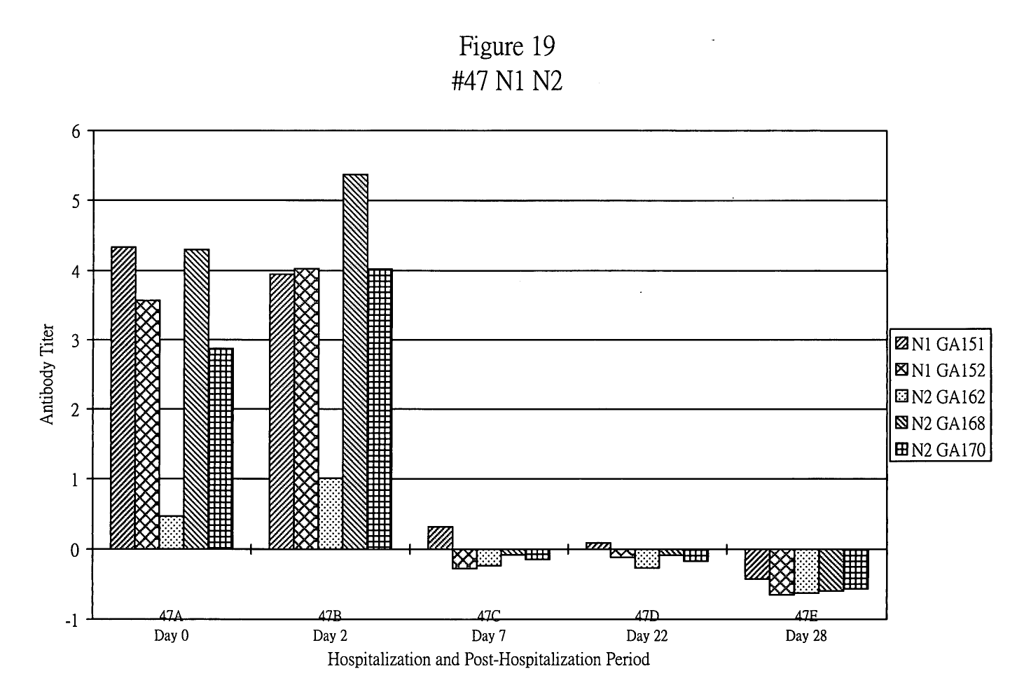

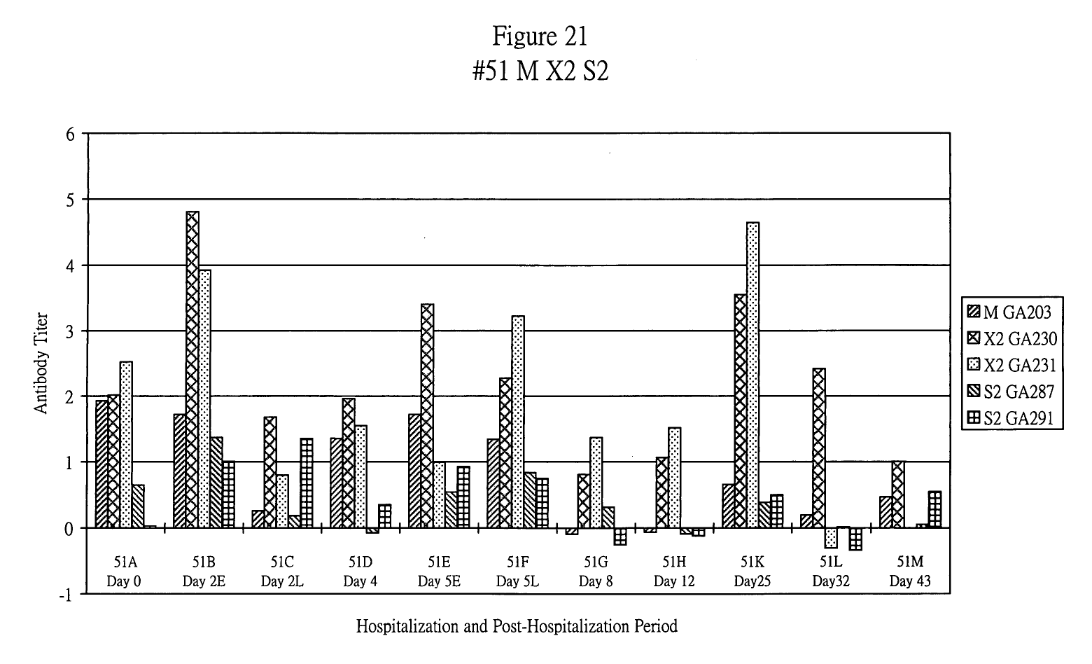

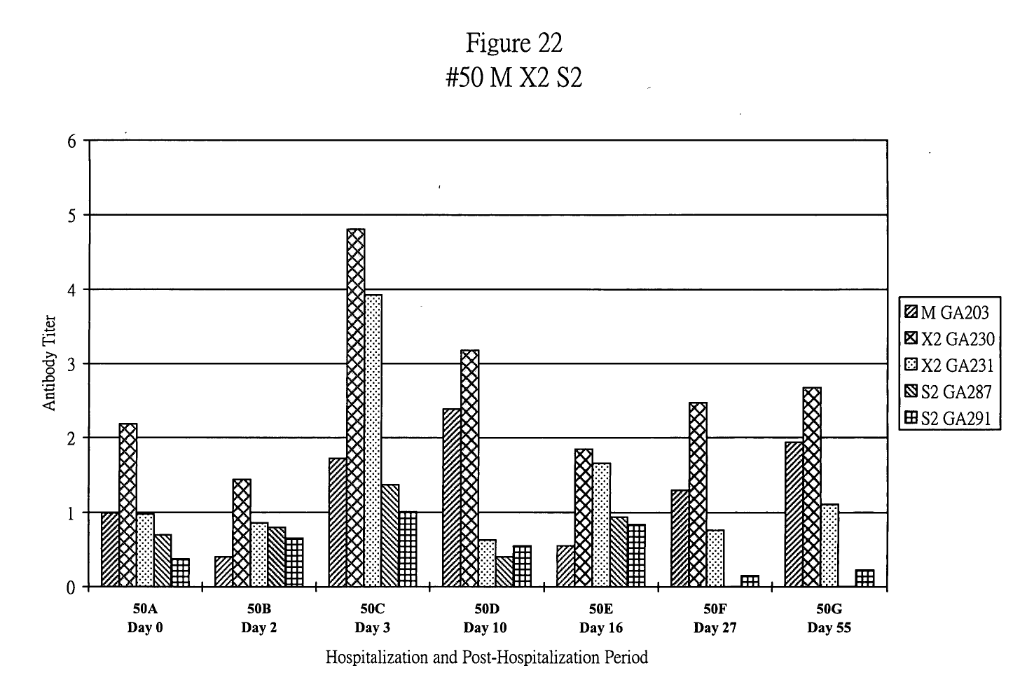

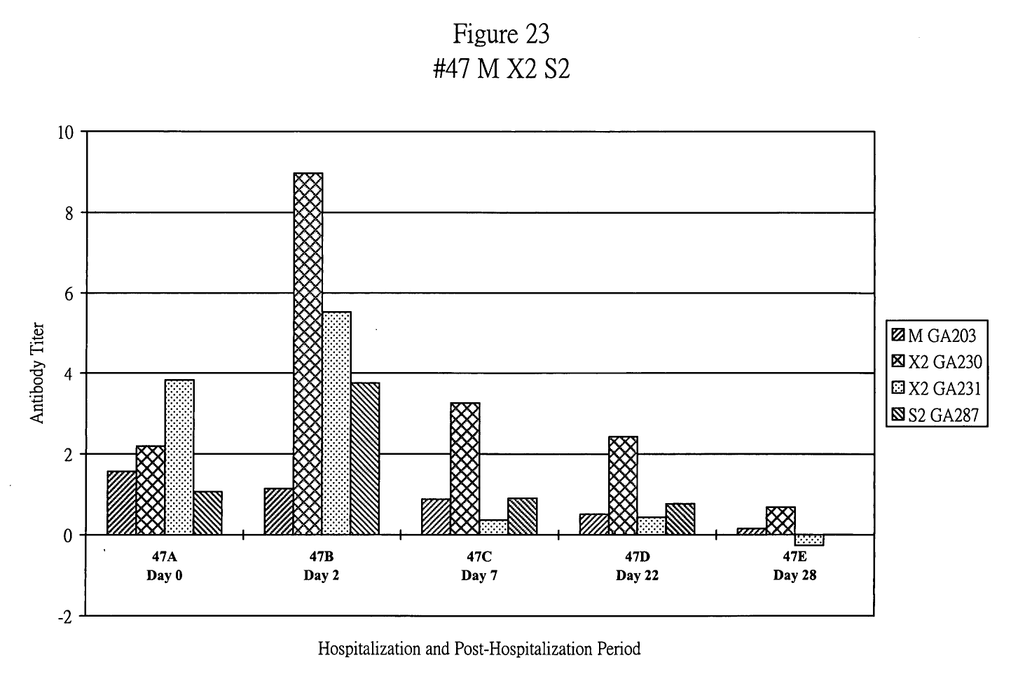

The sera of three probable SARS patients (#51, #50, and #47) were analyzed at multiple time points using the most specific epitopes (N11: GA151, GA152, N2: GA162, GA168, GA170, M: GA203, X2: GA230, GA231, S2: GA287, GA291) as markers. Then, the time-dependent epitope profiles of probable SARS patients' anti-SARS-CoV sera (see FIGS. 17, 18, 19, 21, 22, and 23) were ascertained. First, there were quick and fluctuating immune responses to SARS-CoV within the same day in the probable SARS patients (#51B and #51C, #51E and #51F) (see FIGS. 17 and 21). Also, the phenomena of higher antibody titer during the hospital treatment period, in contrast to the lower antibody titer in the recovery period after discharge from the hospital, was significant in the time-dependent epitope profile analysis, #51K versus #51L, #50C versus #50D, #47B versus #47C (see FIGS. 17, 18, 19, 21, 22, and 23). Moreover, by using the most specific epitope profile in ELISA method, the anti-SARS-CoV immune response can be detected two days earlier than the RT-PCR method of virus RNA detection, used with sample #51C (see patient #51's patient record in Example 4). For example, using ELISA with epitopes GA151, GA152, GA168, GA170, GA203. GA230, and GA231, SARS-COV antibody can be detected in the SARS probable patient #51 on day 0 (sample #51A) (see FIGS. 17 and 21).

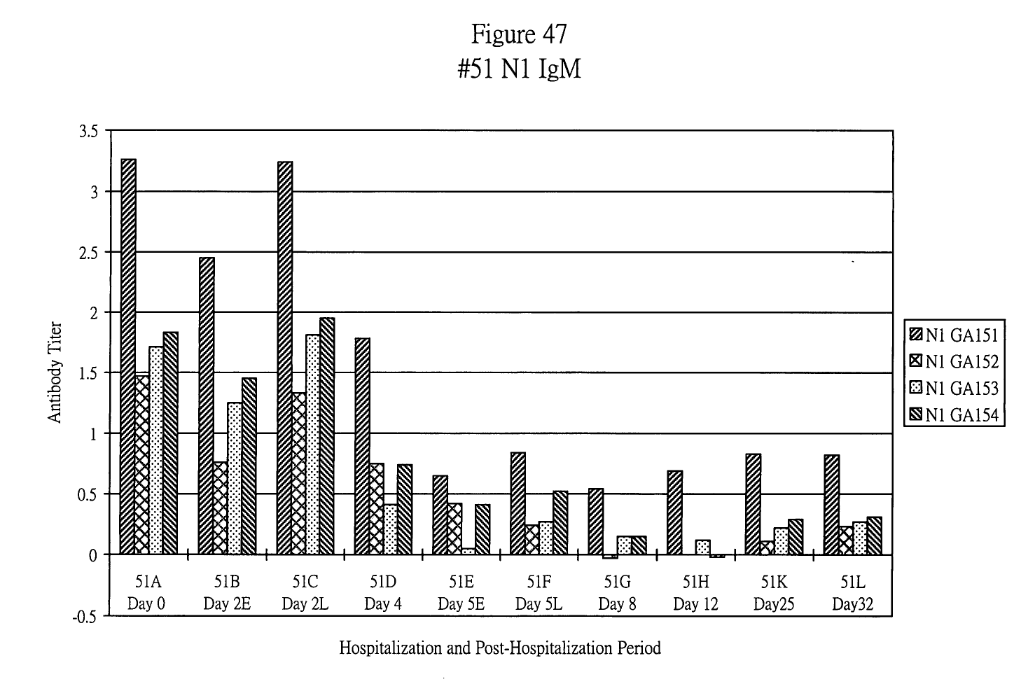

In a comparative analysis of SARS-probable patient's IgM response to SARS-COV infection from early to late stage (other analysis were of patients' IgG response), we used the most specific epitopes (N1: GA151, GA152, GA153, GA154) as markers to analyze one probable SARS patients' sera (#51) at multiple time points. The higher AT of IgM specific to SARS-COV N1 was detected only in the early stage (#51 day 0 (R/T=4/4), day 2E (R/T=3/4), day 2L (R/T=414), and day 4 (R/T=1/4)), but not in the later stage of hospitalization (day 5˜day 25) nor during the recovery period after discharge from hospital (day 32) (see FIG. 47). The phenomena of higher antibody titer in the late hospital therapeutic period, in contrast to lower antibody titer in the recovery period after discharge from the hospital, was not observed in the IgM response but was present in the corresponding IgG response. Again, by using the specific epitope profile in the ELISA method, the anti-SARS-COV immune response of IgM can be detected two days earlier than the RT-PCR method of virus RNA detection used with sample #51C. For example, using the ELISA with epitopes GA151, GA152, GA153, and GA154, SARS-COV antibody can be detected in the SARS-probable patient #51 on day 0 (sample #51A) (see FIG. 47). The antibody titer of human IgM specific to SARS-COV protein (such as the nucleocapsid) is at least 10 times less than that of the corresponding IgG counterpart. The assay of IgM has to be carried out with AT at 300× dilution, as AT would be negative at 3000× dilution. On the other hand, IgG immune response assay can be carried with AT at 3000× dilution (see FIG. 17 and FIG. 47).

In a parallel analysis of epitope profile of serum obtained at different time points, the time dependent epitopes were identified. The SARS-COV infected patients' antibody bound the immediate early (days 1-6) epitopes: X2: 124-138 (GA230, GA231); S2: 1136-1145, 1156-1165 (GA287, GA291); M: 95-104 (GA203); N1: 145-154 (GA151), 150-159 (GA152); N2: 365-374 (GA168), 375-384 (GA170); and S2: 1156-1165 (GA291) and early (days 7-29) epitope: N2: 335-344 (GA162), (see FIGS. 17, 18, 19, 21, 22, and 23).

By ELISA analysis of SARS patients' serum at different time points, the SARS-CoV specific epitopes were further characterized by their time dependency in the period of infection and recovery.

### Example 7

### Cross Reaction of Epitope Profiles with Chicken Anti-Avian Infectious Bronchitis Virus (IBV) and Cat Anti-Feline Coronavirus Sera

To confirm the profile of the SARS-CoV epitopes profile established based on the use of SARS-COV infected human sera, ELISAs were performed with chicken anti-avian infectious bronchitis virus sera and cat anti-feline coronavirus sera.

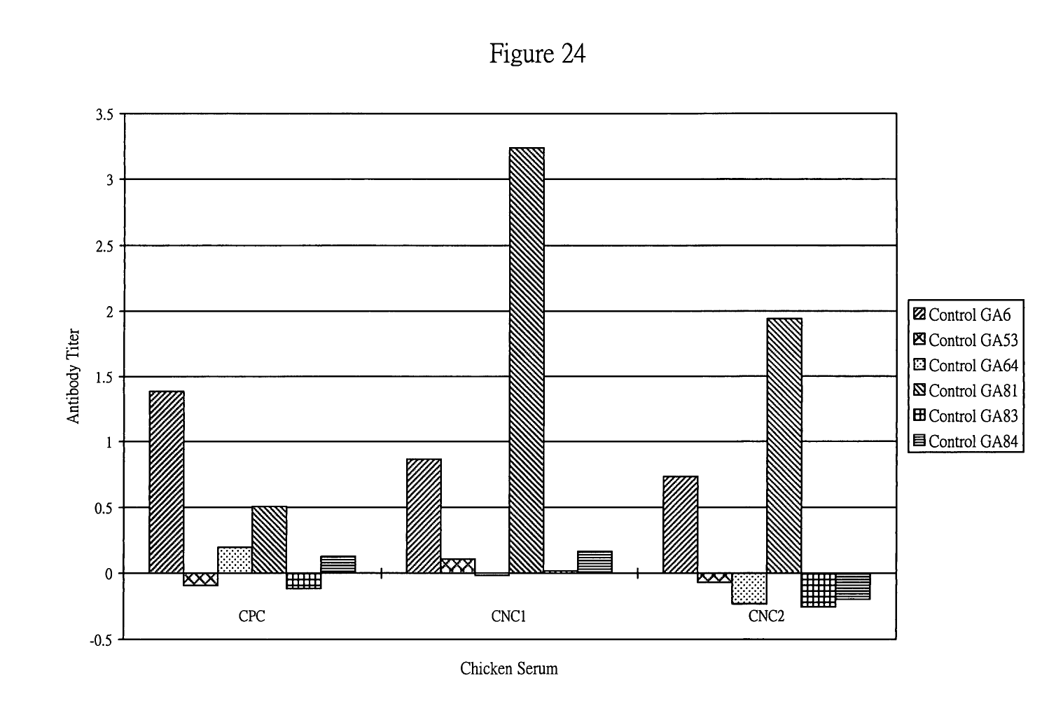

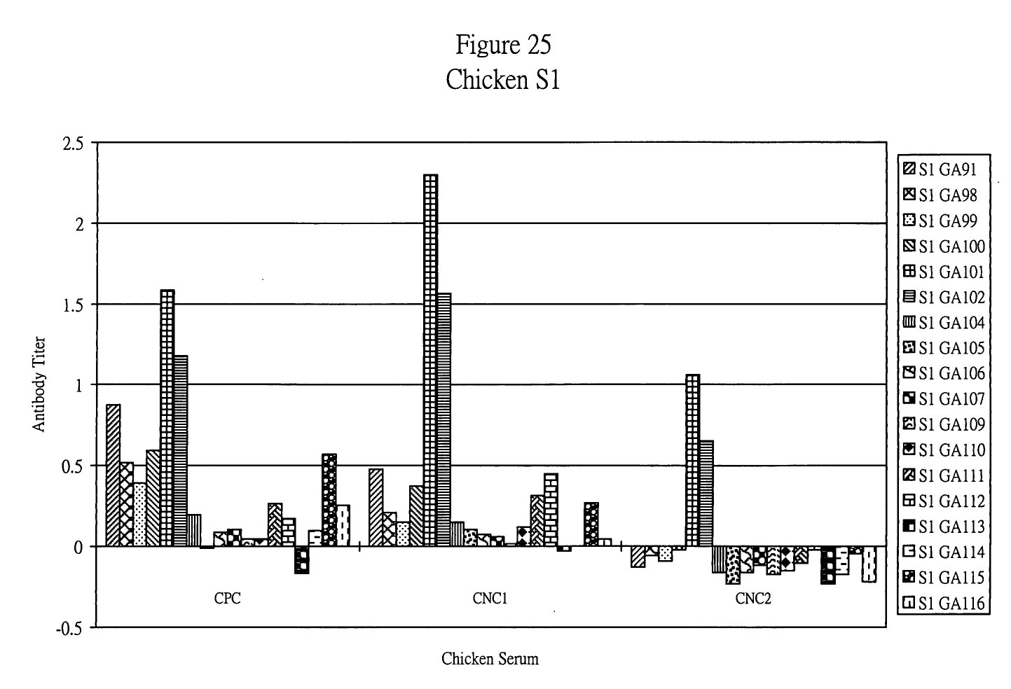

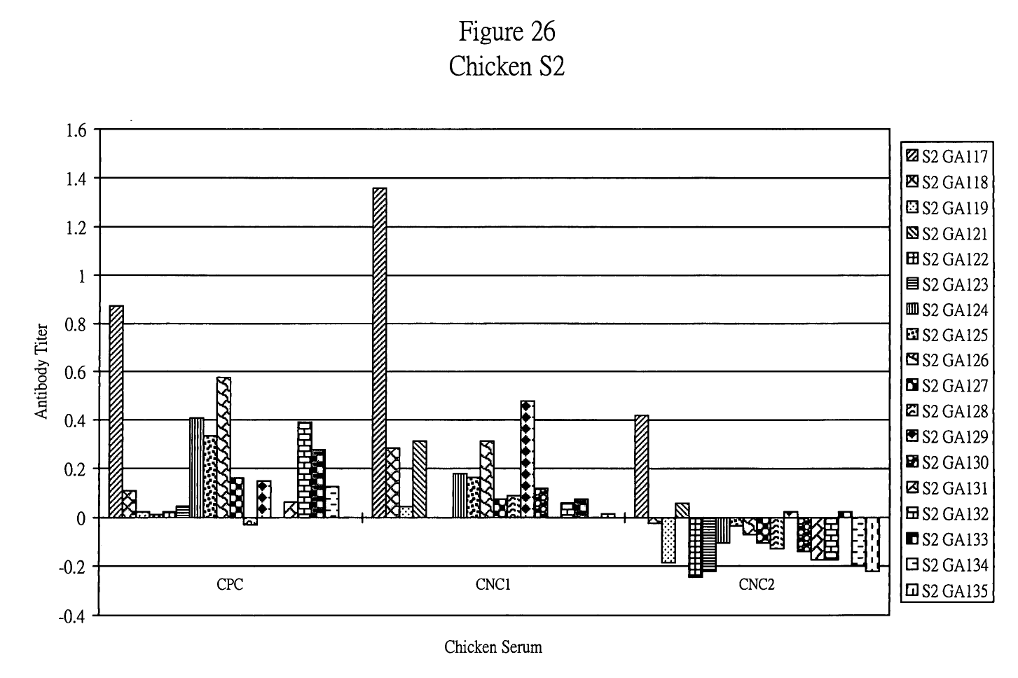

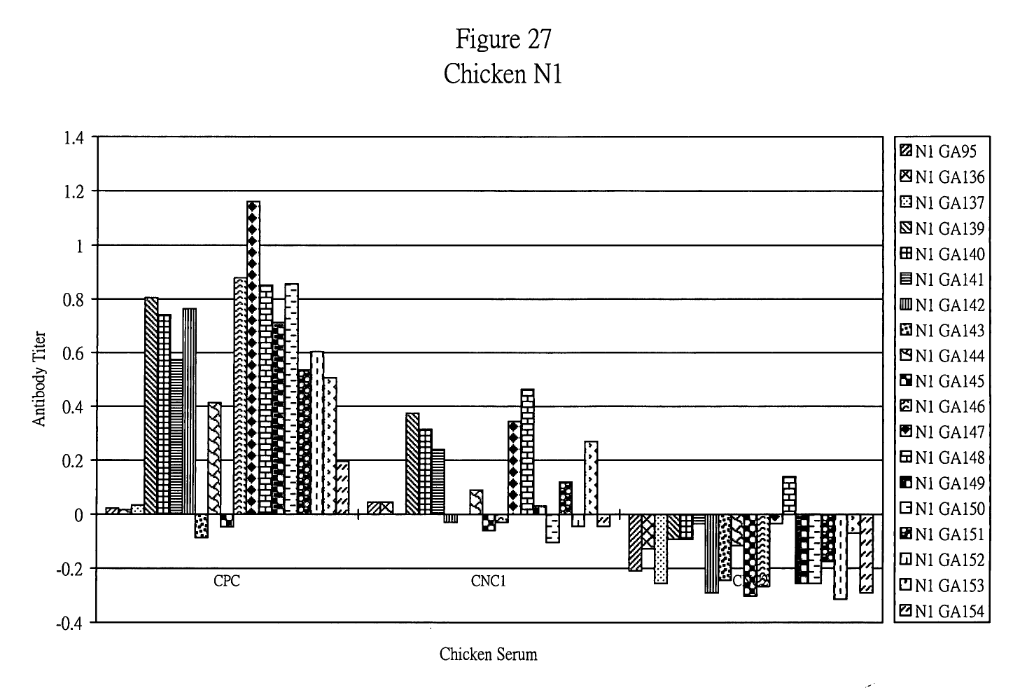

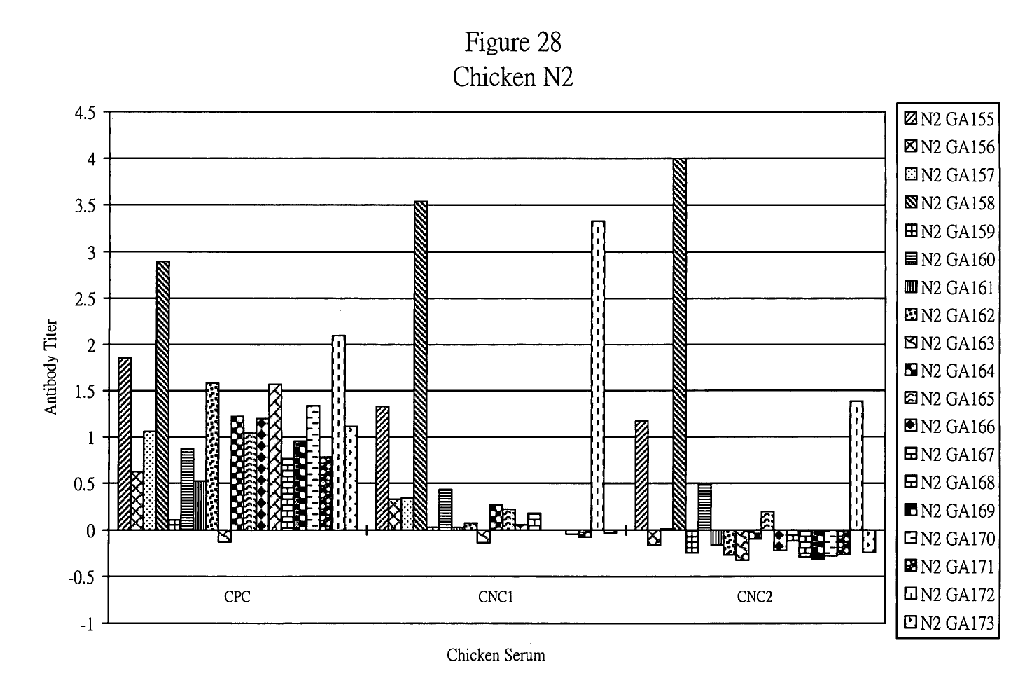

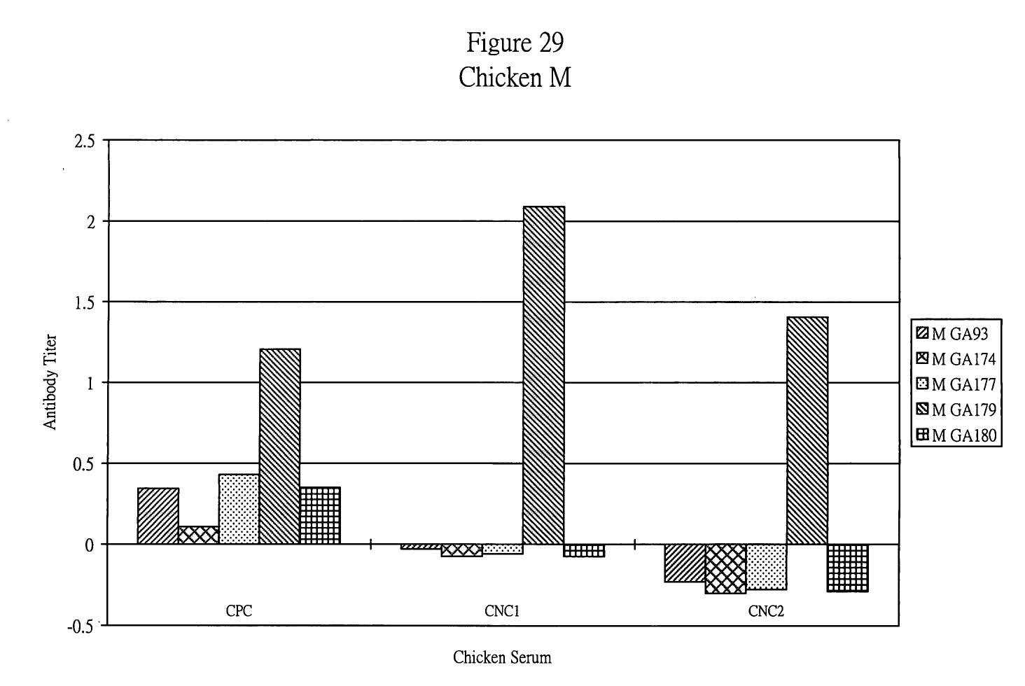

Three chicken sera samples were used in ELISA (2 SPF sera (CNC1 and CNC2) and 1 anti-IBV positive serum (CPC)). The chicken anti-IBV antiserum cross reacted only but weakly with GA115 (S1), GA170 (N2), GA173 (N2), and GA186 (protein X1), but did not cross react with the GA151 and GA152 epitopes which were good markers for detecting SARS-COV infected sera. The non-specific epitopes frequently cross reacted with the chicken sera. Epitope GA6 (WSSV) had a response ratio of R/T=1/3, with a binding result in the CPC serum (see FIG. 24). Epitope GA101 (S1) had a response ratio of R/T=3/3, with the strongest binding activity in the CNC1 serum (see FIG. 25). Epitope GA102 (S1) had a response ratio of R/T=2/3, with a binding activity in CPC and CNC1 sera (see FIG. 25) Epitope GA117 (S2) had a response ratio of R/T=1/3, with a binding activity in the CNC1 serum (see FIG. 26). Epitope GA147 (N2) had a response ratio of R/T=1/3, with a binding activity in the CPC serum (see FIG. 27). GA147 is a cross-species coronavirus specific epitope that is recognized by antibodies against human coronavirus as well as IBV. Epitope GA155 (N2) had a response ratio of R/T=2/3, with a binding activity in the CNC1, CNC2 and CPC sera (see FIG. 28). Epitope GA158 (N2) had a response ratio of R/T=3/3, with binding activities in all three chicken sera (see FIG. 28). Epitope GA172 (N2) had a response ratio of R/T=3/3, with binding activities in all three chicken sera (see FIG. 28). Epitope GA179 (M2) had a response ratio of R/T=3/3, with binding activities in all three chicken sera (see FIG. 29).

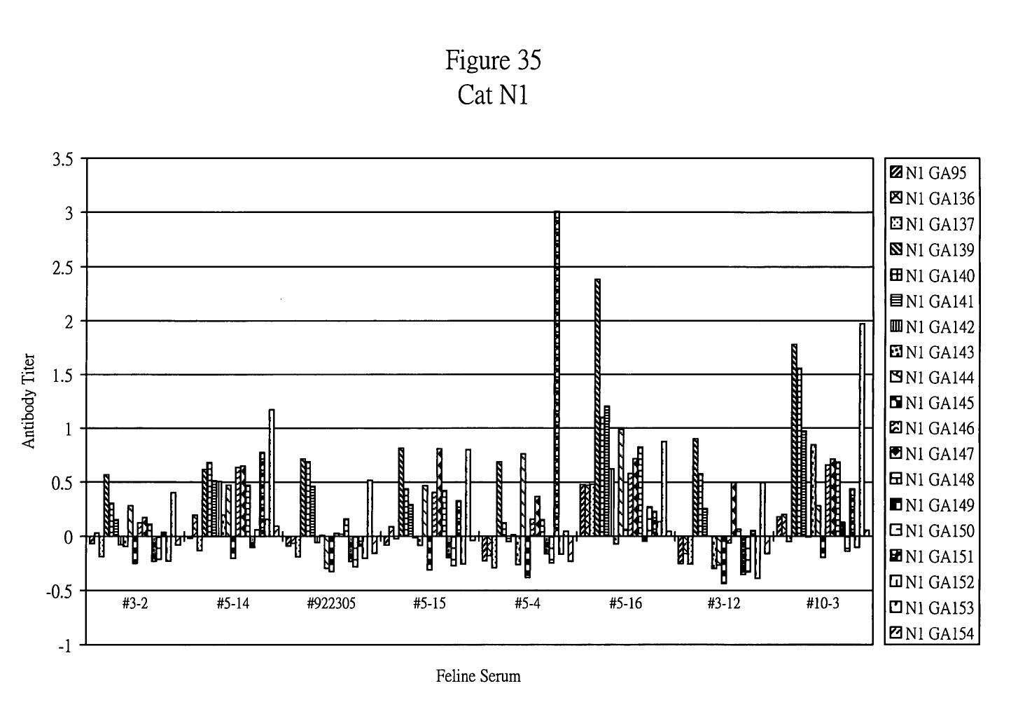

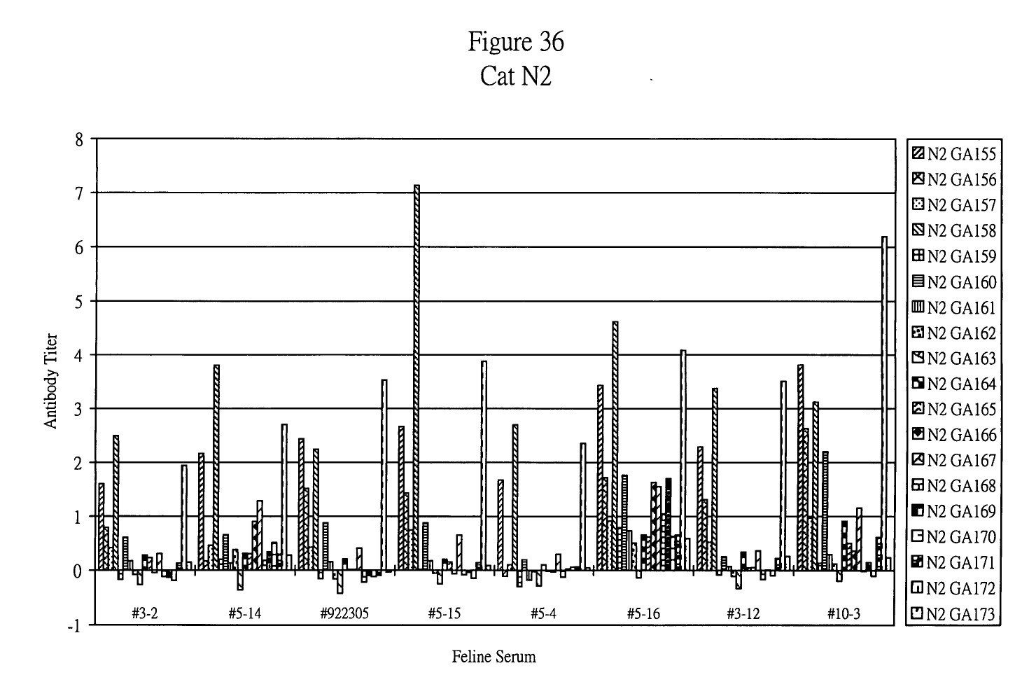

The cat anti-feline coronavirus (FCV) sera interaction with these polypeptides was also analyzed using 8 sera samples (2 negative FCV antibody rapid test sera #5-14, #3-2; 5 positive FCV antibody rapid test sera #54, #5-16, #3-12, #10-3, #5-15; and 1 sera with not test performed #922305). The result was R/T=0 (AbT<1) in all 8 sera with the SARS related specific epitopes GA152 (R/T=0/8) (see FIG. 35) and GA170 (R/T=0/8) (see FIG. 36). Only #5-4 cat serum cross reacted with the specific epitope of GA151 (AbT=2.9) (R/T=1/8) (see FIG. 35).

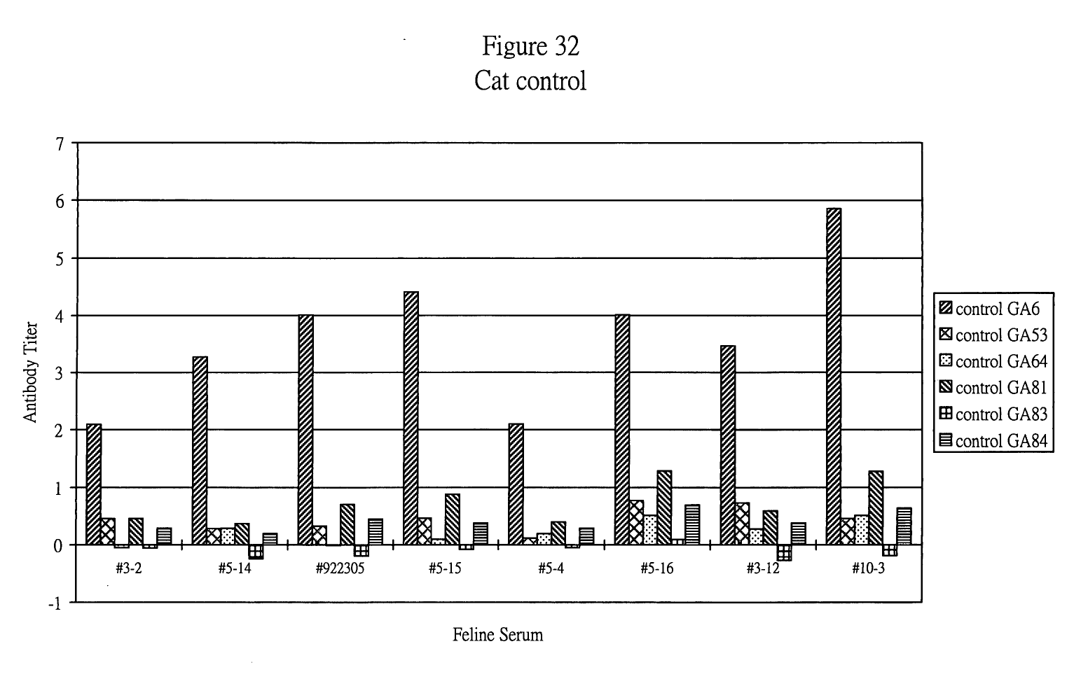

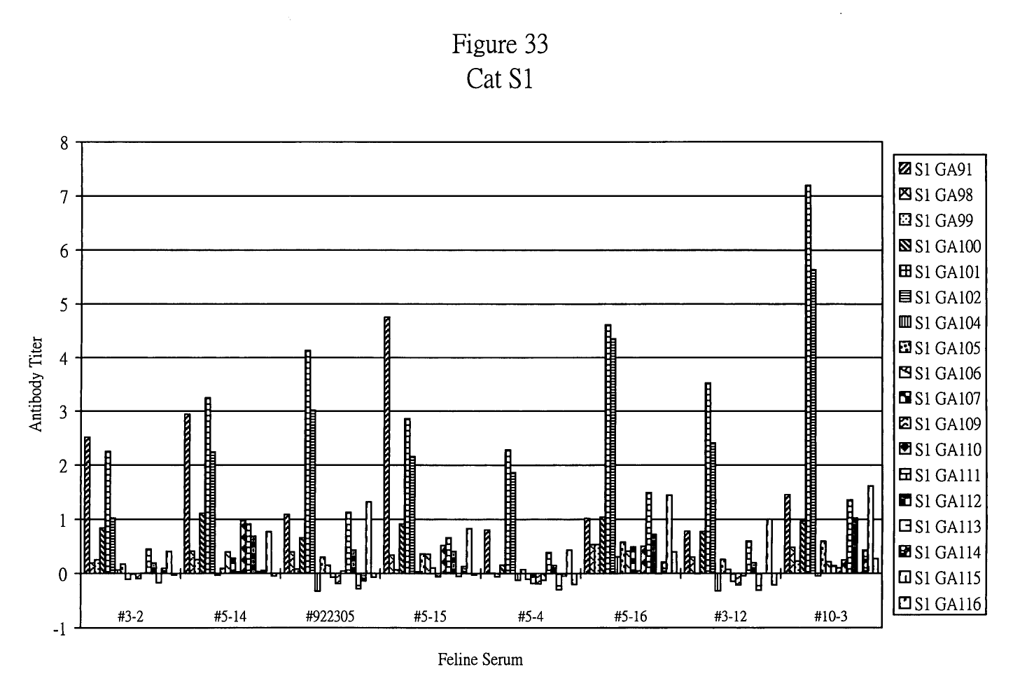

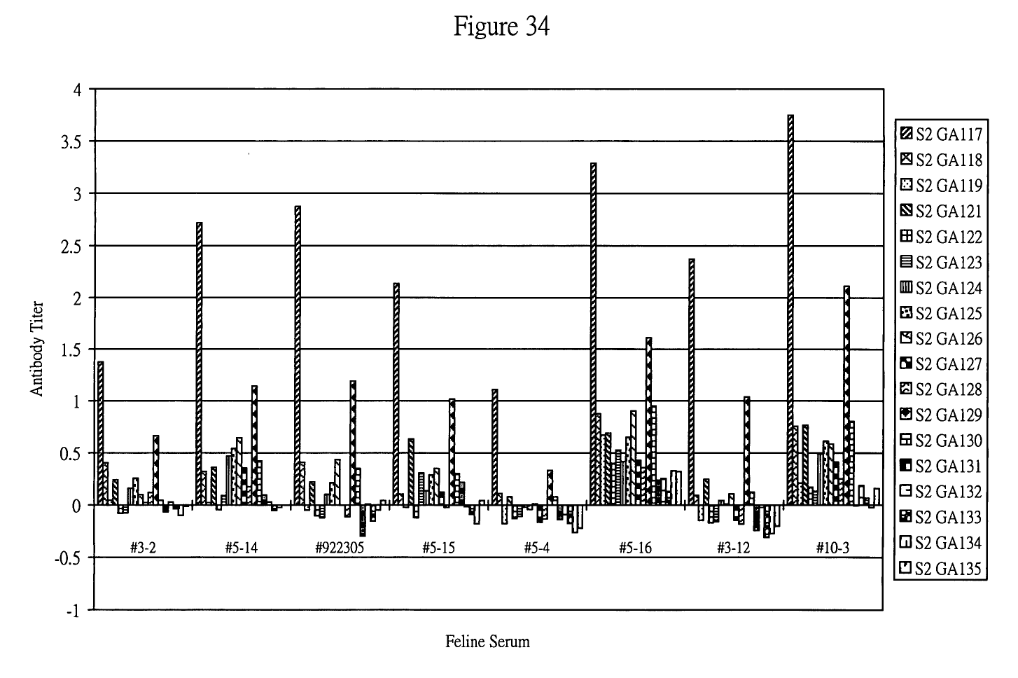

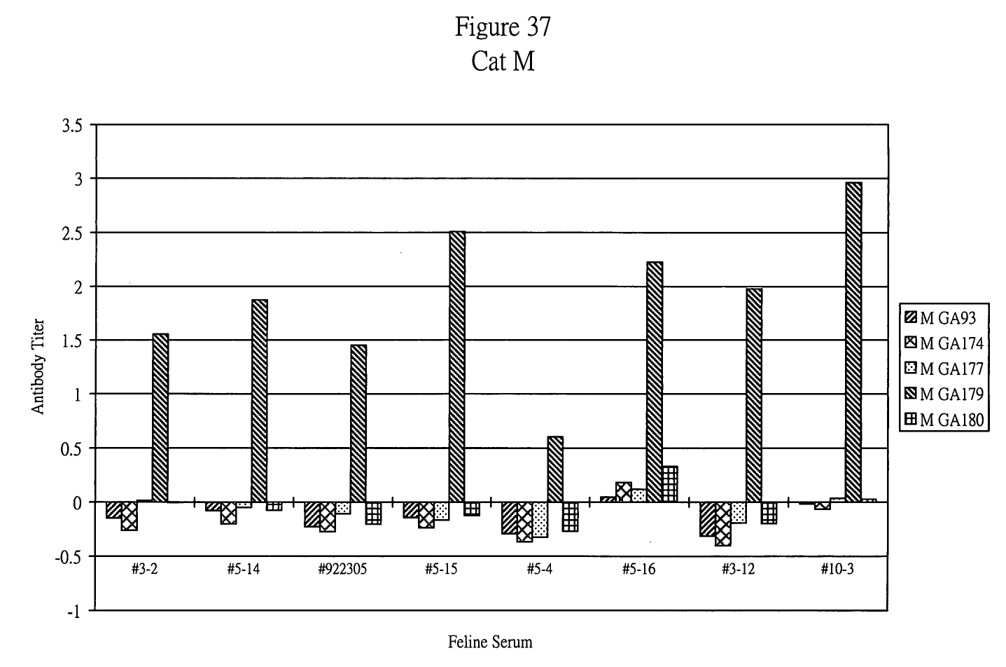

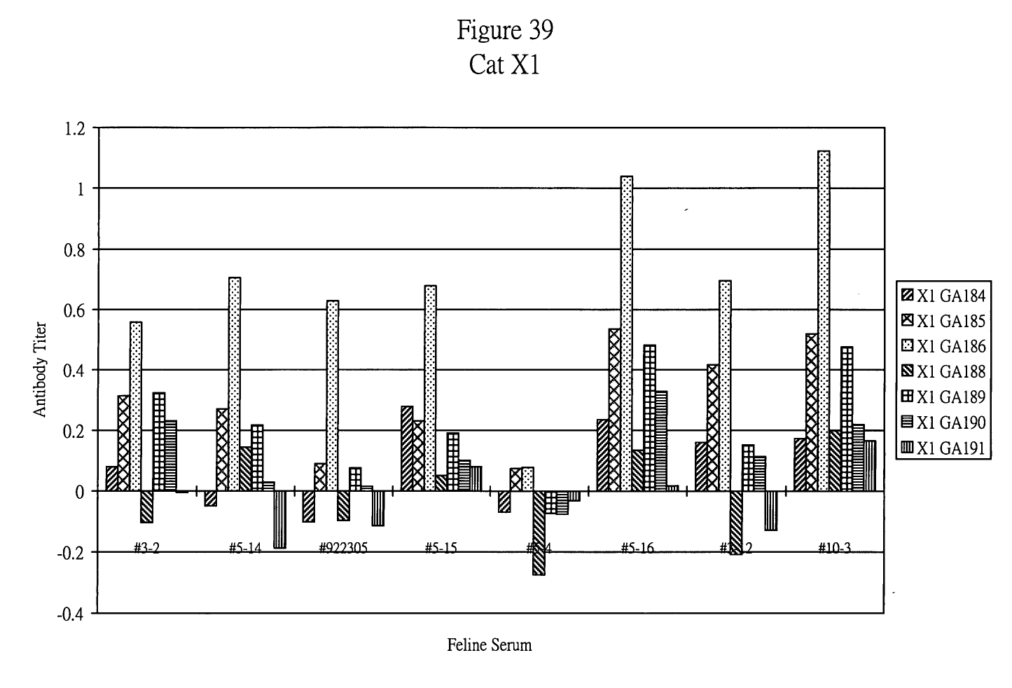

Sera samples (where FCV antibody rapid test was positive and where rapid test was not performed), cross reacted with SARS-CoV epitope GA129 (AbT>1) except for the #5-4 cat sample. The response ratio was R/T 5/6. The non-specific epitopes frequently cross reacted with the cat sera. Epitope GA6 (WSSV) had a response ratio of R/T=8/8, with binding activities in all eight sera samples (see FIG. 32). Epitope GA91 (S1) had a response ratio of R/T=6/8 (see FIG. 39). Epitope GA101 (S1) had a response ratio of R/T=8/8, with binding activities in all eight sera samples (see FIG. 33). Epitope GA102 (S1) had a response ratio of R/T=8/8, with binding activities in all eight sera samples (see FIG. 33). Epitope GA117 (S2) had a response ratio of R/T=6/8 (see FIG. 34). Epitope GA155 (N2) had a response ratio of R/T=8/8 (see FIG. 36). Epitope GA158 (N2) had a response ratio of R/T=8/8 (see FIG. 36). Epitope GA172 (N2) had a response ratio of R/T=8/8 (see FIG. 36). Epitope GA179 (M2) had a response ratio of R/T=7/8, with negative response in sample #5-4) (see FIG. 37).

The SARS-COV related specific epitopes did not interact with the chicken IBV nor the FCV sera, confirming the specificity of these epitopes to SARS-Coronavirus.

### Example 8

### Production of Antibodies Specific to Epitopes

Branched multiple antigenic peptides were used as immunogens to raise corresponding antibodies in animal hosts. Each peptide of GA91˜GA96, GA98˜GA191 was used as an immunogen to raise corresponding antibody in mice, rabbits, and ducks. Four doses were administered in 50 days.

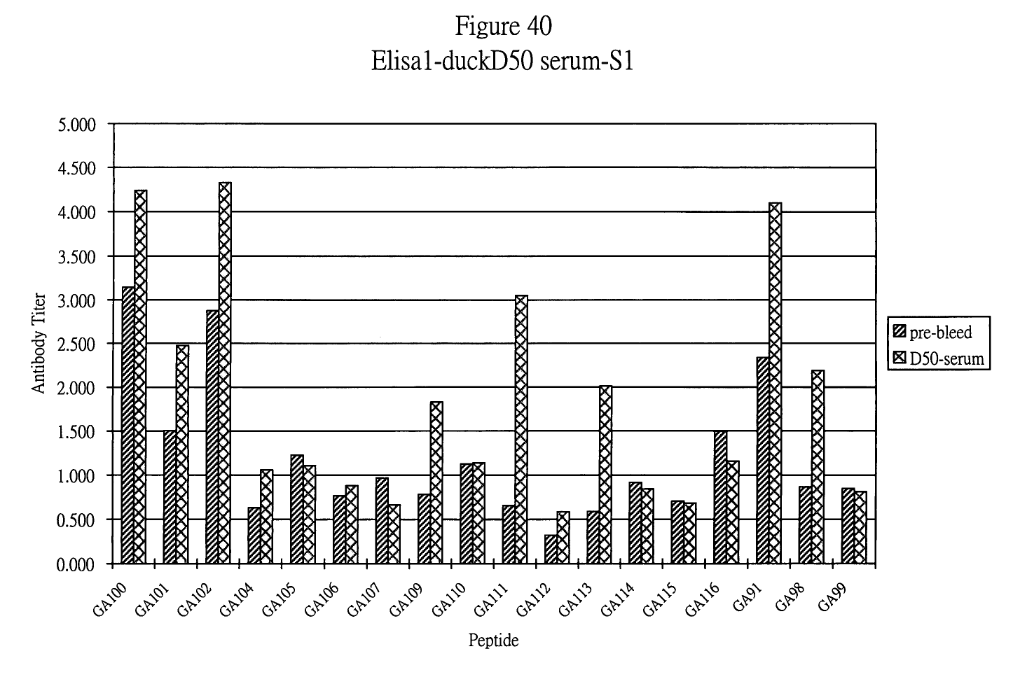

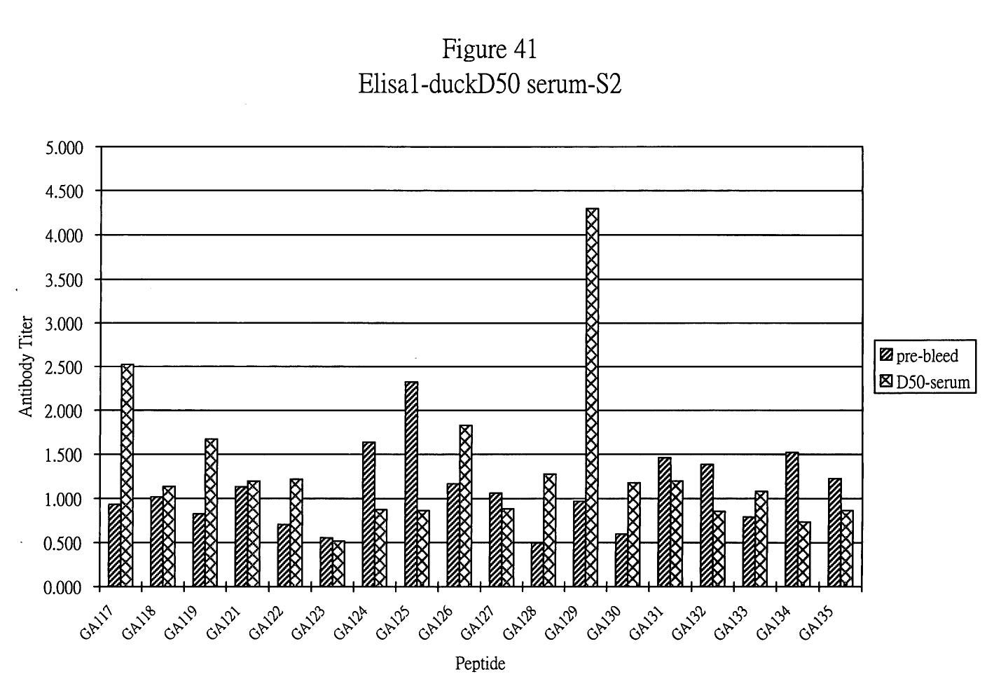

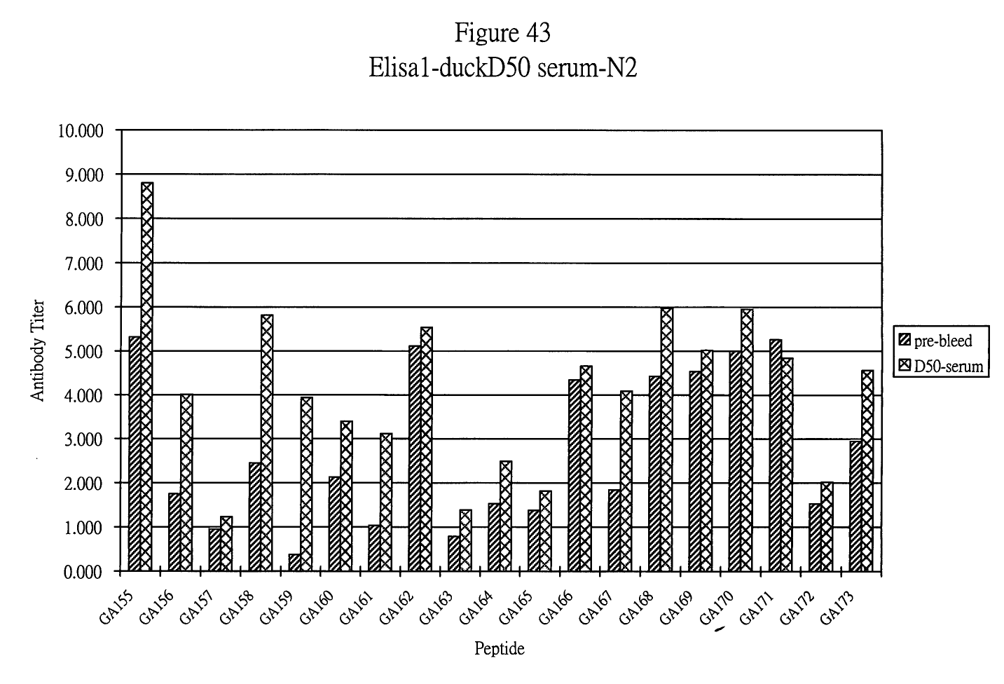

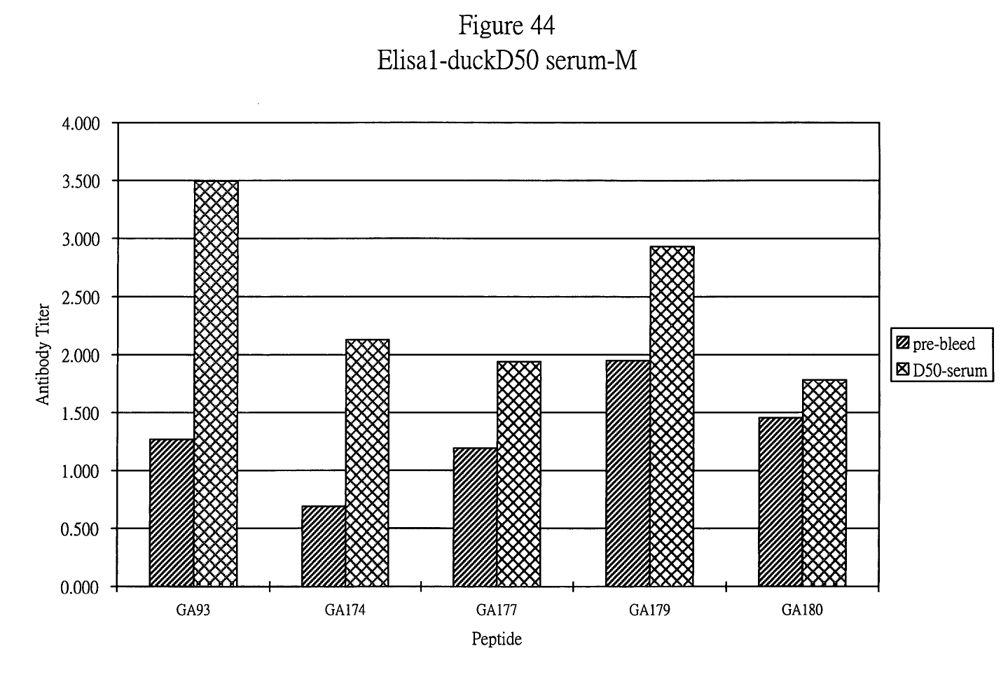

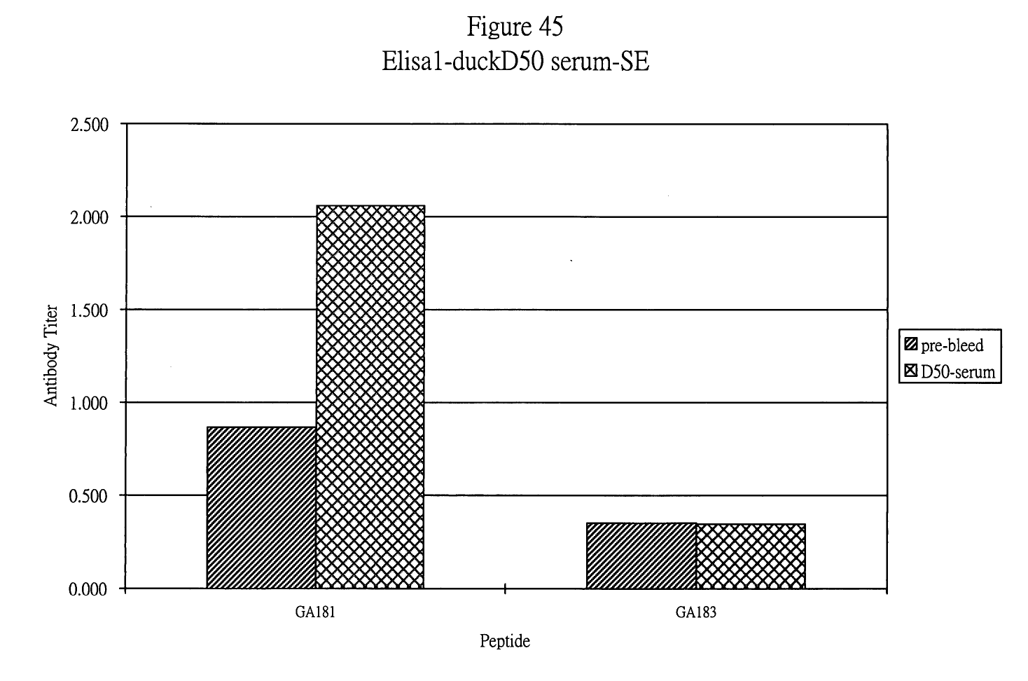

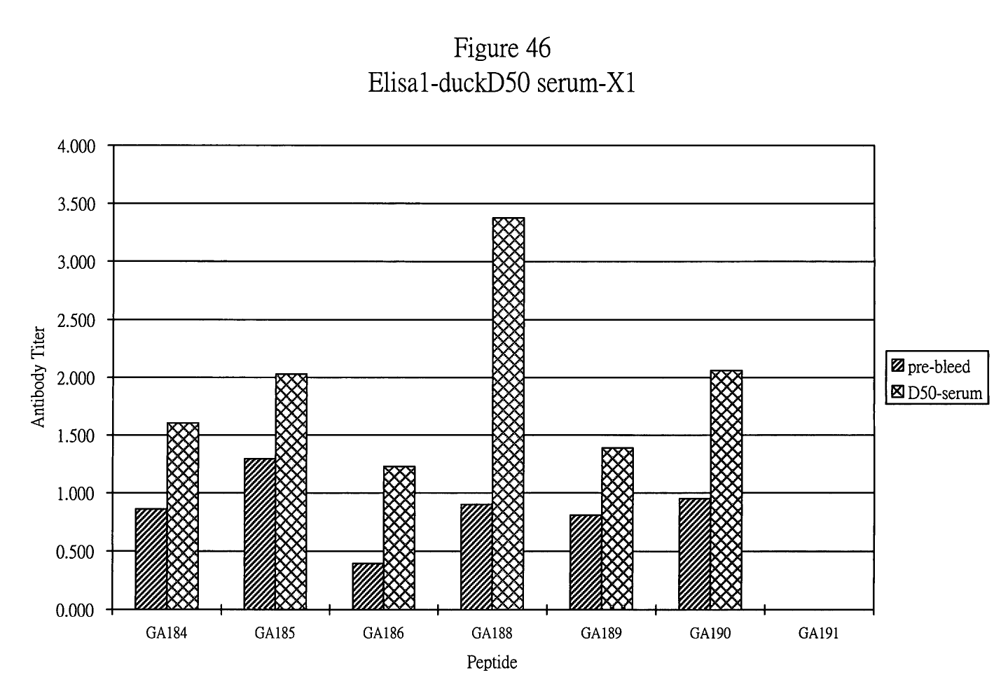

The same immunogenic peptides were used to test immune response. In ducks, a high immune response was detected using epitopes of S1: 431-450 (GA100˜GA102), 437-461 (GA91), 476-505 (GA109, GA111, GA13), as shown in FIG. 40; S2: 1021-1040 (GA117, GA119), 1076-1095 (GA128-GA130), as shown in FIG. 41; N1: 105-124 (GA143, GA145), 107-126 (GA95), 140-169 (GA150˜GA154), as shown in 5C; N2: 300-344 (GA155, GA156, GA158˜GA161), 329-350 (GA96), 360-369 (GA167), 390-399 (GA173), as shown in FIG. 43; M: 1-10 (GA174), 4-20 (GA93), as shown in FIG. 44; sE: 1-10 (GA181), as shown in FIG. 45; and protein X1: 11-40 (GA186, GA188˜GA190), as shown in FIG. 46.

Thus, polyclonal antibodies can be produced by immunizing animal hosts with these SARS-CoV epitopes.

TABLE 1

| | | | | | | | |
|---|---|---|---|---|---|---|---|
| | SEQ ID | | | | | | |
| Zone | NO. | Peptide | Sequence | Location | Annotation | Format | Notes |

| | | | | | | | |
|---|---|---|---|---|---|---|---|
| Control | 1 | GA6 | (LRIPVTAEVGSSYFK)₈K₄K₂K-βA | 52-66 | WSSV VP28 | M8 | 8 branch MAP |
| | 2 | GA53 | (PKYVKQNTVADVIES)₈K₄K₂K-βA | 306-313/ | Influenza HA1/ | M8 | 8 branch MAP |
| | | | | 410 | EV71 VP1 | | |
| | 3 | GA64 | (VDRAVYWHLKKFAGNA)4K₂K-βA | 274-289 | GNNV coat | M4 | 4 branch MAP |
| | | | | | protein | | |
| | 4 | GA81 | NEIIPSKGCLALYLQQNWWTLLVDLLC—NH2 | 355-364/ | Rabies virus | cyclic | C—C |
| | | | | 157-172 | spike/EBV LMP1 | | disulfide |
| | | | | | | | bridge |
| | 5 | GA83 | LTEVETYVLSIVPSGPLKAEIAQRLEDVF | 4-30 | Matrix protein | linear | linear |
| | | | | | M1 of Influenza | (L) | peptide |
| | | | | | A virus | | |
| | 6 | GA84 | AQRLEDVFAGKNTDLEAYQKRMGVQMQRFK | 25-54 | Matrix protein | L | |
| | | | | | M1 of Influenza | | |
| | | | | | A virus | | |
| S1 | 7 | GA91 | NYKYRYLRHGKLRPFERDISNVPFS | C437-461 | SARS spike (S) | L | |
| | | | | | protein | | |
| | 8 | GA98 | (LAWNTRNIDA)₈K₄K₂K-βA | 421-430 | | M8 | |
| | 9 | GA99 | (RNIDATSTGN)₈K₄K₂K-βA | 426-435 | | M8 | |
| | 10 | GA100 | (TSTGNYNYKY)₈K₄K₂K-βA | 431-440 | | M8 | |
| | 11 | GA101 | (YNYKYRYLRH)₈K₄K₂K-βA | 436-445 | | M8 | |
| | 12 | GA102 | (RYLRHGKLRP)₈K₄K₂K-βA | 441-450 | | M8 | |
| | 13 | GA104 | (FERDISNVPF)₈K₄K₂K-βA | 451-460 | | M8 | |
| | 14 | GA105 | (SNVPFSPDGK)₈K₄K₂K-βA | 456-465 | | M8 | |
| | 15 | GA106 | (SPDGKPCTPP)₈K₄K₂K-βA | 461-470 | | M8 | |
| | 16 | GA107 | (PCTPPALNCY)₈K₄K₂K-βA | 466-475 | | M8 | |
| | 17 | GA109 | (WPLNDYGFYT)₈K₄K₂K-βA | 476-485 | | M8 | |
| | 18 | GA110 | (YGFYTTTGIG)₈K₄K₂K-βA | 481-490 | | M8 | |
| | 19 | GA111 | (TTGIGYQPYR)₈K₄K₂K-βA | 486-495 | | M8 | |
| | 20 | GA112 | (YQPYRVVVLS)₈K₄K₂K-βA | 491-500 | | M8 | |
| | 21 | GA113 | (VVVLSFELLN)₈K₄K₂K-βA | 496-505 | | M8 | |
| | 22 | GA114 | (FELLNAPATV)₈K₄K₂K-βA | 501-510 | | M8 | |
| | 23 | GA115 | (APATVCGPKL)₈K₄K₂K-βA | 506-515 | | M8 | |
| | 24 | GA116 | (GPKLSTDLI)₈K₄K₂K-βA | 511-520 | | M8 | |
| S2 | 25 | GA117 | (RVDFCGKGYH)₈K₄K₂K-βA | 1021-1030 | | M8 | |
| | 26 | GA118 | (GKGYHLMSFP)₈K₄K₂K-βA | 1026-1035 | | M8 | |
| | 27 | GA119 | (LMSFPQAAPH)₈K₄K₂K-βA | 1031-1040 | | M8 | |
| | 28 | GA120 | (QAAPHGVVFL)₈K₄K₂K-βA | 1036-1045 | | M8 | |
| | 29 | GA121 | (GVVFLHVTYV)₈K₄K₂K-βA | 1041-1050 | | M8 | |
| | 30 | GA122 | (HVTYVPSQER)₈K₄K₂K-βA | 1046-1055 | | M8 | |
| | 31 | GA123 | (PSQERNFTTA)₈K₄K₂K-βA | 1051-1060 | | M8 | |
| | 32 | GA124 | (NFTTAPAICH)₈K₄K₂K-βA | 1056-1065 | | M8 | |
| | 33 | GA125 | (PAICHEGKAY)₈K₄K₂K-βA | 1061-1070 | | M8 | |
| | 34 | GA126 | (EGKAYFPREG)₈K₄K₂K-βA | 1066-1075 | | M8 | |
| | 35 | GA127 | (FPREGVFVFN)₈K₄K₂K-βA | 1071-1080 | | M8 | |
| | 36 | GA128 | (VFVFNGTSWF)₈K₄K₂K-βA | 1076-1085 | | M8 | |
| | 37 | GA129 | (GTSWFITQRN)₈K₄K₂K-βA | 1081-1090 | | M8 | |
| | 38 | GA130 | (ITQRNFFSPQ)₈K₄K₂K-βA | 1086-1095 | | M8 | |
| | 39 | GA131 | (FFSPQIITTD)₈K₄K₂K-βA | 1091-1100 | | M8 | |
| | 40 | GA132 | (IITTDNTFVS)₈K₄K₂K-βA | 1096-1105 | | M8 | |
| | 41 | GA133 | (NTFVSGNCDV)₈K₄K₂K-βA | 1101-1110 | | M8 | |
| | 42 | GA134 | (GNCDVVIGII)₈K₄K₂K-βA | 1106-1115 | | M8 | |
| | 43 | GA135 | (VIGIINNTVY)₈K₄K₂K-βA | 1111-1120 | | M8 | |
| N1 | 44 | GA95 | PRWYFYYLGTGPEASLPYGA | C107-126 | SARS nucleo- | L | |
| | | | | | capsid (N) | | |
| | | | | | protein | | |
| | 45 | GA136 | (GQGVPINTNS)₈K₄K₂K-βA | 70-79 | | M8 | |
| | 46 | GA137 | (INTNSGPDDQ)₈K₄K₂K-βA | 75-84 | | M8 | |
| | 195 | GA138 | (GPDDQIGYYR)₈K₄K₂K-βA | 80-89 | | M8 | |
| | 47 | GA139 | (IGYYRRATRR)₈K₄K₂K-βA | 85-94 | | M8 | |
| | 48 | GA140 | (RATRRVRGGD)₈K₄K₂K-βA | 90-99 | | M8 | |
| | 49 | GA141 | (VRGGDGKMKE)₈K₄K₂K-βA | 95-104 | | M8 | |
| | 50 | GA142 | (GKMKELSPRW)₈K₄K₂K-βA | 100-109 | | M8 | |
| | 51 | GA143 | (LSPRWYFYYL)₈K₄K₂K-βA | 105-114 | | M8 | |
| | 52 | GA144 | (YFYYLGTGPE)₈K₄K₂K-βA | 110-119 | | M8 | |
| | 53 | GA145 | (GTGPEASLPY)₈K₄K₂K-βA | 115-124 | | M8 | |
| | 54 | GA146 | (ASLPYGANKE)₈K₄K₂K-βA | 120-129 | | M8 | |
| | 55 | GA147 | (GANKEGIVWV)₈K₄K₂K-βA | 125-134 | | M8 | |
| | 56 | GA148 | (GIVWVATEGA)₈K₄K₂K-βA | 130-139 | | M8 | |
| | 57 | GA149 | (ATEGALNTPK)₈K₄K₂K-βA | 135-144 | | M8 | |
| | 58 | GA150 | (LNTPKDHIGT)₈K₄K₂K-βA | 140-149 | | M8 | |
| | 59 | GA151 | (DHIGTRNPNN)₈K₄K₂K-βA | 145-154 | | M8 | |
| | 60 | GA152 | (RNPNNNAATV)₈K₄K₂K-βA | 150-159 | | M8 | |
| | 61 | GA153 | (NAATVLQLPQ)₈K₄K₂K-βA | 155-164 | | M8 | |
| | 62 | GA154 | (LQLPQGTTLP)₈K₄K₂K-βA | 160-169 | | M8 | |
| | 63 | GA96 | GTWLTYHGAIKLDDKDPQFKDN | C329-350 | | | |
| N2 | 64 | GA155 | (KHWPQIAQFA)₈K₄K₂K-βA | 300-309 | | M8 | |
| | 65 | GA156 | (IAQFAPSASA)₈K₄K₂K-βA | 305-314 | | M8 | |
| | 66 | GA157 | (PSASAFFGMS)₈K₄K₂K-βA | 310-319 | | M8 | |
| | 67 | GA158 | (FFGMSRIGME)₈K₄K₂K-βA | 315-324 | | M8 | |
| | 68 | GA159 | (RIGMEVTPSG)₈K₄K₂K-βA | 320-329 | | M8 | |
| | 69 | GA160 | (VTPSGTWLTY)₈K₄K₂K-βA | 325-334 | | M8 | |
| | 70 | GA161 | (TWLTYHGAIK)₈K₄K₂K-βA | 330-339 | | M8 | |
| | 71 | GA162 | (HGAIKLDDKD)₈K₄K₂K-βA | 335-344 | | M8 | |
| | 72 | GA163 | (LDDKDPQFKD)₈K₄K₂K-βA | 340-349 | | M8 | |
| | 73 | GA164 | (PQFKDNVILL)₈K₄K₂K-βA | 345-354 | | M8 | |
| | 74 | GA165 | (NVILLNKHID)₈K₄K₂K-βA | 350-359 | | M8 | |
| | 75 | GA166 | (NKHIDAYKTF)₈K₄K₂K-βA | 355-364 | | M8 | |
| | 76 | GA167 | (AYKTFPPTEP)₈K₄K₂K-βA | 360-369 | | M8 | |
| | 77 | GA168 | (PPTEPKKDKK)₈K₄K₂K-βA | 365-374 | | M8 | |
| | 78 | GA169 | (KKDKKKKTDE)₈K₄K₂K-βA | 370-379 | | M8 | |
| | 79 | GA170 | (KKTDEAQPLP)₈K₄K₂K-βA | 375-384 | | M8 | |
| | 80 | GA171 | (AQPLPQRQKK)₈K₄K₂K-βA | 380-389 | | M8 | |
| | 81 | GA172 | (QRQKKQPTVT)₈K₄K₂K-βA | 385-394 | | M8 | |
| | 82 | GA173 | (QPTVTLLPAA)₈K₄K₂K-βA | 390-399 | | M8 | |
| M | 83 | GA93 | NGTITVEELKQLLEQWN | C4-20 | SARS matrix (M) | L | |
| | | | | | protein | | |
| | 84 | GA174 | (MADNGTITVE)₈K₄K₂K-βA | 1-10 | | M8 | |
| | 85 | GA175 | (TITVEELKQL)₈K₄K₂K-βA | 6-15 | | M8 | |
| | 86 | GA176 | (ELKQLLEQWN)₈K₄K₂K-βA | 11-20 | | M8 | |
| | 87 | GA177 | (LACFVLAAVY)₈K₄K₂K-βA | 61-70 | | M8 | |
| | 88 | GA179 | (RINWVTGGIA)₈K₄K₂K-βA | 71-80 | | M8 | |
| | 89 | GA180 | (TGGIAIAMAC)₈K₄K₂K-βA | 76-85 | | M8 | |
| sE | 90 | GA181 | (MYSFVSEETG)₈K₄K₂K-βA | 1-10 | SARS small | M8 | |
| | | | | | envelope (sE) | | |
| | | | | | protein | | |
| | 91 | GA182 | (SEETGTLIVN) | 6-15 | | M8 | |
| | 92 | GA183 | (TLIVNSVLLF)₈K₄K₂K-βA | 11-20 | | M8 | |
| X1 | 93 | GA184 | (MDLFMRFFTL)₈K₄K₂K-βA | 1-10 | SARS protein X1 | M8 | |
| | | | | | (X1) | | |
| | 94 | GA185 | (RFFTLGSITA)₈K₄K₂K-βA | 6-15 | | M8 | |
| | 95 | GA186 | (GSITAQPVKI)₈K₄K₂K-βA | 11-20 | | M8 | |
| | 96 | GA187 | (QPVKIDNASP)₈K₄K₂K-βA | 16-25 | | M8 | |
| | 97 | GA188 | (DNASPASTVH)₈K₄K₂K-βA | 21-30 | | M8 | |
| | 98 | GA189 | (ASTVHATATI)₈K₄K₂K-βA | 26-35 | | M8 | |
| | 99 | GA190 | (ATATIPLQAS)₈K₄K₂K-βA | 31-40 | | M8 | |
| | 100 | GA191 | (HLLLVAAGME)₈K₄K₂K-βA | 93-102 | | M8 | |
| sE | 101 | GA192 | SVLLFLAFVV | 16-25 | SARS small | L | |
| | | | | | envelope (sE) | | |
| | | | | | protein | | |
| | 102 | GA193 | LAFVVFLLVT | 21-30 | | L | |
| | 103 | GA194 | FLLVTLAILT | 26-35 | | L | |
| | 104 | GA195 | LAILTALRLC | 31-40 | | L | |
| | 105 | GA196 | ALRLCAYCCN | 36-45 | | L | |
| | 106 | GA197 | AYCCNIVNVS | 41-50 | | L | |
| | 107 | GA198 | IVNVSLVKPT | 46-55 | | L | |
| | 108 | GA199 | LVKPTVYVYS | 51-60 | | L | |
| | 109 | GA200 | VYVYSRVKNL | 56-65 | | L | |
| | 110 | GA201 | RVKNLNSSEG | 61-70 | | L | |
| | 111 | GA202 | SSEGVPDLLV | 67-76 | | L | |
| M | 112 | GA203 | FVASFRLFAR | 95-104 | SARS matrix (M) | L | |
| | | | | | protein | | |
| | 113 | GA204 | RLFARTRSMW | 100-109 | | L | |
| | 114 | GA205 | TRSMWSFNPE | 105-114 | | L | |
| | 115 | GA206 | SFNPETNILL | 110-119 | | L | |
| | 116 | GA207 | TNILLNVPLR | 115-124 | | L | |
| | 117 | GA208 | PKEITVATSR | 164-173 | | L | |
| | 118 | GA209 | VATSRTLSYY | 169-178 | | L | |
| | 119 | GA210 | TLSYYKLGAS | 174-183 | | L | |
| | 120 | GA211 | KLGASQRVGT | 179-188 | | L | |
| | 121 | GA212 | QRVGTDSGFA | 184-193 | | L | |
| | 122 | GA213 | DSGFAAYNRY | 189-198 | | L | |
| | 123 | GA214 | AYNRYRIGNY | 194-203 | | L | |
| X2 | 124 | GA215 | QIQLSLLKVT | 24-33 | SARS protein X2 | L | |
| | | | | | (X2) | | |
| | 125 | GA216 | LLKVTAFQHQ | 29-38 | | L | |
| | 126 | GA217 | AFQHQNSKKT | 34-43 | | L | |
| | 127 | GA218 | NSKKTTKLVV | 39-48 | | L | |
| | 128 | GA219 | TKLVVILRIG | 44-53 | | L | |
| | 129 | GA220 | ILRIGTQVLK | 49-58 | | L | |
| | 130 | GA221 | TQVLKTMSLY | 54-63 | | L | |
| | 131 | GA222 | TMSLYMAISP | 59-68 | | L | |
| | 132 | GA223 | MAISPKFTTS | 64-73 | | L | |
| | 133 | GA224 | KFTTSLSLHK | 69-78 | | L | |
| | 134 | GA225 | LSLHKLLQTL | 74-83 | | L | |
| | 135 | GA226 | LLQTLVLKML | 79-88 | | L | |
| | 136 | GA227 | VLKMLHSSSL | 84-93 | | L | |
| | 137 | GA228 | HSSSLTSLLK | 89-98 | | L | |
| | 138 | GA229 | WIQFMMSRRR | 119-128 | | L | |
| | 139 | GA230 | MSRRRLLACL | 124-133 | | L | |
| | 140 | GA231 | LLACLCKHKK | 129-138 | | L | |
| | 141 | GA232 | KHKKVSTNL | 134-143 | | L | |
| | 142 | GA233 | STNLCTHSFR | 140-149 | | L | |
| X3 | 143 | GA234 | MFHLVDFQVT | 1-10 | SARS protein X3 | L | |
| | | | | | (X3) | | |
| | 144 | GA235 | DFQVTIAEIL | 6-15 | | L | |
| | 145 | GA236 | IAEILIIIMR | 11-20 | | L | |
| | 146 | GA237 | IIIMRTFRIA | 16-25 | | L | |
| | 147 | GA238 | TFRIAIWNLD | 21-30 | | L | |
| | 148 | GA239 | IWNLDVIISS | 26-35 | | L | |
| | 149 | GA240 | VIISSIVRQL | 31-40 | | L | |
| | 150 | GA241 | IVRQLFKPLT | 36-45 | | L | |
| | 151 | GA242 | FKPLTKKNYS | 41-50 | | L | |
| | 152 | GA243 | KKNYSELDDE | 46-55 | | L | |
| | 153 | GA244 | DEEPMELDYP | 54-63 | | L | |
| X4 | 154 | GA245 | GTRHTYQLRA | 70-79 | SARS protein X4 | L | |
| | | | | | (X4) | | |
| | 155 | GA246 | YQLRARSVSP | 75-84 | | L | |
| | 156 | GA247 | RSVSPKLFIR | 80-89 | | L | |
| | 157 | GA248 | KLFIRQEEVQ | 85-94 | | L | |
| | 158 | GA249 | QEEVQQELYS | 90-99 | | L | |
| | 159 | GA250 | QELYSPLFLI | 95-104 | | L | |
| | 160 | GA251 | PLFLIVAALV | 100-109 | | L | |
| | 161 | GA252 | VAALVFLILC | 105-114 | | L | |
| | 162 | GA253 | FLILCFTIKR | 110-119 | | L | |
| X5 | 163 | GA254 | MCLKILVRYN | 1-10 | SARS protein X5 | L | |
| | | | | | (X5) | | |
| | 164 | GA255 | LVRYNTRGNT | 6-15 | | L | |
| | 165 | GA256 | TRGNTYSTAW | 11-20 | | L | |
| | 166 | GA257 | YSTAWLCALG | 16-25 | | L | |
| | 167 | GA258 | LCALGKVLPF | 21-30 | | L | |
| | 168 | GA259 | KVLPFHRWHT | 26-35 | | L | |
| | 169 | GA260 | HRWHTMVQTC | 31-40 | | L | |
| | 170 | GA261 | MVQTCTPNVT | 36-45 | | L | |
| | 171 | GA262 | TPNVTINCQD | 41-50 | | L | |
| | 172 | GA263 | INCQDPAGGA | 46-55 | | L | |
| | 173 | GA264 | PAGGALIARC | 51-60 | | L | |
| | 174 | GA265 | LIARCWYLHE | 56-65 | | L | |
| | 175 | GA266 | WYLHEGHQTA | 61-70 | | L | |
| | 176 | GA267 | GHQTAAFRDV | 66-75 | | L | |
| | 177 | GA268 | AFRDVLVVLN | 71-80 | | L | |
| | 178 | GA269 | VLVVLNKRTN | 75-84 | | L | |
| S2 | 179 | GA283 | (NNTVYDPLQP)₈K₄K₂K-βA | 1116-1125 | SARS spike (S) | M8 | |
| | | | | | protein | | |
| | 180 | GA284 | (DPLQPELDSF)₈K₄K₂K-βA | 1121-1130 | | M8 | |
| | 181 | GA285 | (ELDSFKEELD)₈K₄K₂K-βA | 1126-1135 | | M8 | |
| | 182 | GA286 | (KEELDKYFKN)₈K₄K₂K-βA | 1131-1140 | | M8 | |
| | 183 | GA287 | (KYFKNHTSPD)₈K₄K₂K-βA | 1136-1145 | | M8 | |
| | 184 | GA288 | (HTSPDVDLGD)₈K₄K₂K-βA | 1141-1150 | | M8 | |
| | 185 | GA289 | (VDLGDISGIN)₈K₄K₂K-βA | 1146-1155 | | M8 | |
| | 186 | GA290 | (ISGINASVVN)₈K₄K₂K-βA | 1151-1160 | | M8 | |
| | 187 | GA291 | (ASVVNIQKEI)₈K₄K₂K-βA | 1156-1165 | | M8 | |
| | 188 | GA292 | (IQKEIDRLNE)₈K₄K₂K-βA | 1161-1170 | | M8 | |
| | 189 | GA293 | (DRLNEVAKNL)₈K₄K₂K-βA | 1166-1175 | | M8 | |
| | 190 | GA294 | (VAKNLNESLI)₈K₄K₂K-βA | 1171-1180 | | M8 | |
| | 191 | GA295 | (NESLIDLQEL)₈K₄K₂K-βA | 1176-1185 | | M8 | |
| | 192 | GA296 | (DLQELGKYEQ)₈K₄K₂K-βA | 1181-1190 | | M8 | |
| | 193 | GA297 | (GKYEQYIKWP)₈K₄K₂K-βA | 1186-1195 | | M8 | |
| | 194 | GA298 | (YIKWPWYVWL)₈K₄K₂K-βA | 1191-1200 | | M8 | |

1. One or more polypeptide(s) comprising amino acid sequence(s) selected from SEQ ID NO: 40, SEQ ID NO: 42, SEQ ID NO: 46, SEQ ID NO: 47, SEQ ID NO:50, SEQ ID NO: 54, SEQ ID NO: 55, SEQ ID NO: 57, SEQ ID NO: 58, SEQ ID NO: 59, SEQ ID NO: 60, SEQ ID NO: 61, SEQ ID NO: 62, SEQ ID NO: 65, SEQ ID NO: 69, SEQ ID NO: 70, SEQ ID NO: 71, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 112, SEQ ID NO: 139, SEQ ID NO: 140, SEQ ID NO: 156, SEQ ID NO: 183, and SEQ ID NO: 187 and degenerate variant(s) of thereof.

2. One or more polypeptide(s) comprising amino acid sequence(s) selected from SEQ ID NO: 11, SEQ ID NO: 12, SEQ ID NO: 25, SEQ ID NO: 64, SEQ ID NO: 67, SEQ ID NO: 81, SEQ ID NO: 88, and SEQ ID NO: 118 and degenerate variant(s) thereof.

3. One or more polypeptide(s) comprising amino acid sequence(s) selected from SEQ ID NO: 11, SEQ ID NO: 12, SEQ ID NO: 25, SEQ ID NO: 40, SEQ ID NO: 42, SEQ ID NO: 46, SEQ ID NO: 49, SEQ ID NO: 50, SEQ ID NO: 51, SEQ ID NO: 54, SEQ ID NO: 55, SEQ ID NO: 57, SEQ ID NO: 58, SEQ ID NO: 59, SEQ ID NO: 60, SEQ ID NO: 61, SEQ ID NO: 62, SEQ ID NO: 64, SEQ ID NO: 65, SEQ ID NO: 67, SEQ ID NO: 69, SEQ ID NO: 70, SEQ ID NO: 71, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 88, and SEQ ID NO: 140 and degenerate variant(s) thereof.

4. One or more polypeptide(s) comprising amino acid sequence(s) selected from SEQ ID NO: 1, SEQ ID NO: 2, SEQ ID NO: 3, SEQ ID NO: 4, SEQ ID NO: 5, and SEQ ID NO: 6 and degenerate variant(s) thereof.

5. An immunogenic composition that comprises one or more polypeptides comprising amino acid sequences selected from SEQ ID NO: 7, SEQ ID NO: 11, SEQ ID NO: 12, SEQ ID NO: 17, SEQ ID NO: 19, SEQ ID NO: 21, SEQ ID NO: 25, SEQ ID NO: 27, SEQ ID NO: 36, SEQ ID NO: 37, SEQ ID NO: 38, SEQ ID NO: 44, SEQ ID NO: 51, SEQ ID NO: 53, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 67, SEQ ID NO: 68, SEQ ID NO: 69, SEQ ID NO: 70, SEQ ID NO: 76, SEQ ID NO: 82, SEQ ID NO: 83, SEQ ID NO: 84, SEQ ID NO: 90, SEQ ID NO: 95, SEQ ID NO: 97, SEQ ID NO: 98, and SEQ ID NO: 99 and degenerate variant(s) thereof.

6. The polypeptide of any of claims 1, 2, 3, 4, or 5, wherein the polypeptide is in a linear form.

7. The polypeptide of any of claims 1, 2, 3, 4, or 5, wherein the polypeptide is in a branched form.

8. An apparatus bearing one or more polypeptide(s) comprising amino acid sequence(s) selected from SEQ ID NO: 7-SEQ ID NO: 195 and the degenerate variants thereof.

9. An apparatus bearing one or more polypeptide(s) comprising amino acid sequence(s) selected from SEQ ID NO: 44-SEQ ID NO: 62 and the degenerate variants thereof.

10. An apparatus bearing one or more polypeptide(s) comprising amino acid sequence(s) selected from SEQ ID NO: 63-SEQ ID NO: 82 and the degenerate variants thereof.

11. An apparatus bearing one or more polypeptide(s) comprising amino acid sequence(s) selected from SEQ ID NO: 124-SEQ ID NO: 142 and the degenerate variants thereof.

12. An apparatus bearing one or more polypeptide(s) comprising amino acid sequence(s) selected from SEQ ID NO: 83-SEQ ID NO: 89 and SEQ ID NO: 112-SEQ ID NO: 123 and the degenerate variants thereof.

13. An apparatus bearing one or more polypeptide(s) comprising amino acid sequence(s) selected from SEQ ID NO: 7-SEQ ID NO: 43 and the degenerate variants thereof.

14. An apparatus bearing one or more polypeptide(s) comprising amino acid sequence(s) selected from SEQ ID NO: 59, SEQ ID NO: 60, and SEQ ID NO: 79 and the degenerate variants thereof.

15. An apparatus bearing one or more polypeptide(s) comprising amino acid sequence(s) selected from SEQ ID NO: 59, SEQ ID NO: 60, SEQ ID NO: 77, SEQ ID NO: 79, SEQ ID NO: 112, SEQ ID NO: 139, SEQ ID NO: 140, SEQ ID NO: 183, and SEQ ID NO: 187 and the degenerate variants thereof.

16. An apparatus bearing a polypeptide comprising amino acid sequence of SEQ ID NO: 71 and the degenerate variants thereof.

17. The degenerate variant(s) of claims 1, 2, 3, 4, 5, 8, 9, 10, 11, 12, 13, 14, 15 or 16, wherein the one or more different amino acid(s) in the degenerate variant(s) have the same hydrophobic/hydrophilic nature as the original amino acid(s).

18. The degenerate variant(s) of claims 1, 2, 3, 4, 5, 8, 9, 10, 11, 12, 13, 14, 15 or 16, wherein the one or more different amino acid(s) in the degenerate variant(s) have the same +/−charge(s) as the original amino acid(s).


SEQUENCE LISTING (195 sequences)

SEQ ID NO: 1 - protein, 15 aa, Shrimp White Spot Syndrome Virus
LRIPVTAEVGSSYFK

SEQ ID NO: 2 - protein, 15 aa, Artificial Sequence
  Description of Artificial Sequence hemagglutinin of Influenza A Virus fused with VP1 of enterovirus 71
PKYVKQNTVADVIES

SEQ ID NO: 3 - protein, 16 aa, Fish Nervous Necrosis Virus
VDRAVYWHLKKFAGNA

SEQ ID NO: 4 - protein, 27 aa, Artificial Sequence
  Description of Artificial Sequence spike glycoprotein of rabies virus fused with peptide sequence of latent membrane protein LMP-1 of human herpesvirus 4/Epstein-Barr Virus
NEIIPSKGCLALYLQQNWWTLLVDLLC

SEQ ID NO: 5 - protein, 29 aa, Influenza A Virus
LTEVETYVLSIVPSGPLKAEIAQRLEDVF

SEQ ID NO: 6 - protein, 30 aa, Influenza A Virus
AQRLEDVFAGKNTDLEAYQKRMGVQMQRFK

SEQ ID NO: 7 - protein, 25 aa, SARS Coronavirus
NYKYRYLRHGKLRPFERDISNVPFS

SEQ ID NO: 8 - protein, 10 aa, SARS Coronavirus
LAWNTRNIDA

SEQ ID NO: 9 - protein, 10 aa, SARS Coronavirus
RNIDATSTGN

SEQ ID NO: 10 - protein, 10 aa, SARS Coronavirus
TSTGNYNYKY

SEQ ID NO: 11 - protein, 10 aa, SARS Coronavirus
YNYKYRYLRH

SEQ ID NO: 12 - protein, 10 aa, SARS Coronavirus
RYLRHGKLRP

SEQ ID NO: 13 - protein, 10 aa, SARS Coronavirus
FERDISNVPF

SEQ ID NO: 14 - protein, 10 aa, SARS Coronavirus
SNVPFSPDGK

SEQ ID NO: 15 - protein, 10 aa, SARS Coronavirus
SPDGKPCTPP

SEQ ID NO: 16 - protein, 10 aa, SARS Coronavirus
PCTPPALNCY

SEQ ID NO: 17 - protein, 10 aa, SARS Coronavirus
WPLNDYGFYT

SEQ ID NO: 18 - protein, 10 aa, SARS Coronavirus
YGFYTTTGIG

SEQ ID NO: 19 - protein, 10 aa, SARS Coronavirus
TTGIGYQPYR

SEQ ID NO: 20 - protein, 10 aa, SARS Coronavirus
YQPYRVVVLS

SEQ ID NO: 21 - protein, 10 aa, SARS Coronavirus
VVVLSFELLN

SEQ ID NO: 22 - protein, 10 aa, SARS Coronavirus
FELLNAPATV

SEQ ID NO: 23 - protein, 10 aa, SARS Coronavirus
APATVCGPKL

SEQ ID NO: 24 - protein, 9 aa, SARS Coronavirus
GPKLSTDLI

SEQ ID NO: 25 - protein, 10 aa, SARS Coronavirus
RVDFCGKGYH

SEQ ID NO: 26 - protein, 10 aa, SARS Coronavirus
GKGYHLMSFP

SEQ ID NO: 27 - protein, 10 aa, SARS Coronavirus
LMSFPQAAPH

SEQ ID NO: 28 - protein, 10 aa, SARS Coronavirus
QAAPHGVVFL

SEQ ID NO: 29 - protein, 10 aa, SARS Coronavirus
GVVFLHVTYV

SEQ ID NO: 30 - protein, 10 aa, SARS Coronavirus
HVTYVPSQER

SEQ ID NO: 31 - protein, 10 aa, SARS Coronavirus
PSQERNFTTA

SEQ ID NO: 32 - protein, 10 aa, SARS Coronavirus
NFTTAPAICH

SEQ ID NO: 33 - protein, 10 aa, SARS Coronavirus
PAICHEGKAY

SEQ ID NO: 34 - protein, 10 aa, SARS Coronavirus
EGKAYFPREG

SEQ ID NO: 35 - protein, 10 aa, SARS Coronavirus
FPREGVFVFN

SEQ ID NO: 36 - protein, 10 aa, SARS Coronavirus
VFVFNGTSWF

SEQ ID NO: 37 - protein, 10 aa, SARS Coronavirus
GTSWFITQRN

SEQ ID NO: 38 - protein, 10 aa, SARS Coronavirus
ITQRNFFSPQ

SEQ ID NO: 39 - protein, 10 aa, SARS Coronavirus
FFSPQIITTD

SEQ ID NO: 40 - protein, 10 aa, SARS Coronavirus
IITTDNTFVS

SEQ ID NO: 41 - protein, 10 aa, SARS Coronavirus
NTFVSGNCDV

SEQ ID NO: 42 - protein, 10 aa, SARS Coronavirus
GNCDVVIGII

SEQ ID NO: 43 - protein, 10 aa, SARS Coronavirus
VIGIINNTVY

SEQ ID NO: 44 - protein, 20 aa, SARS Coronavirus
PRWYFYYLGTGPEASLPYGA

SEQ ID NO: 45 - protein, 10 aa, SARS Coronavirus
GQGVPINTNS

SEQ ID NO: 46 - protein, 10 aa, SARS Coronavirus
INTNSGPDDQ

SEQ ID NO: 47 - protein, 10 aa, SARS Coronavirus
IGYYRRATRR

SEQ ID NO: 48 - protein, 10 aa, SARS Coronavirus
RATRRVRGGD

SEQ ID NO: 49 - protein, 10 aa, SARS Coronavirus
VRGGDGKMKE

SEQ ID NO: 50 - protein, 10 aa, SARS Coronavirus
GKMKELSPRW

SEQ ID NO: 51 - protein, 10 aa, SARS Coronavirus
LSPRWYFYYL

SEQ ID NO: 52 - protein, 10 aa, SARS Coronavirus
YFYYLGTGPE

SEQ ID NO: 53 - protein, 10 aa, SARS Coronavirus
GTGPEASLPY

SEQ ID NO: 54 - protein, 10 aa, SARS Coronavirus
ASLPYGANKE

SEQ ID NO: 55 - protein, 10 aa, SARS Coronavirus
GANKEGIVWV

SEQ ID NO: 56 - protein, 10 aa, SARS Coronavirus
GIVWVATEGA

SEQ ID NO: 57 - protein, 10 aa, SARS Coronavirus
ATEGALNTPK

SEQ ID NO: 58 - protein, 10 aa, SARS Coronavirus
LNTPKDHIGT

SEQ ID NO: 59 - protein, 10 aa, SARS Coronavirus
DHIGTRNPNN

SEQ ID NO: 60 - protein, 10 aa, SARS Coronavirus
RNPNNNAATV

SEQ ID NO: 61 - protein, 10 aa, SARS Coronavirus
NAATVLQLPQ

SEQ ID NO: 62 - protein, 10 aa, SARS Coronavirus
LQLPQGTTLP

SEQ ID NO: 63 - protein, 22 aa, SARS Coronavirus
GTWLTYHGAIKLDDKDPQFKDN

SEQ ID NO: 64 - protein, 10 aa, SARS Coronavirus
KHWPQIAQFA

SEQ ID NO: 65 - protein, 10 aa, SARS Coronavirus
IAQFAPSASA

SEQ ID NO: 66 - protein, 10 aa, SARS Coronavirus
PSASAFFGMS

SEQ ID NO: 67 - protein, 10 aa, SARS Coronavirus
FFGMSRIGME

SEQ ID NO: 68 - protein, 10 aa, SARS Coronavirus
RIGMEVTPSG

SEQ ID NO: 69 - protein, 10 aa, SARS Coronavirus
VTPSGTWLTY

SEQ ID NO: 70 - protein, 10 aa, SARS Coronavirus
TWLTYHGAIK

SEQ ID NO: 71 - protein, 10 aa, SARS Coronavirus
HGAIKLDDKD

SEQ ID NO: 72 - protein, 10 aa, SARS Coronavirus
LDDKDPQFKD

SEQ ID NO: 73 - protein, 10 aa, SARS Coronavirus
PQFKDNVILL

SEQ ID NO: 74 - protein, 10 aa, SARS Coronavirus
NVILLNKHID

SEQ ID NO: 75 - protein, 10 aa, SARS Coronavirus
NKHIDAYKTF

SEQ ID NO: 76 - protein, 10 aa, SARS Coronavirus
AYKTFPPTEP

SEQ ID NO: 77 - protein, 10 aa, SARS Coronavirus
PPTEPKKDKK

SEQ ID NO: 78 - protein, 10 aa, SARS Coronavirus
KKDKKKKTDE

SEQ ID NO: 79 - protein, 10 aa, SARS Coronavirus
KKTDEAQPLP

SEQ ID NO: 80 - protein, 10 aa, SARS Coronavirus
AQPLPQRQKK

SEQ ID NO: 81 - protein, 10 aa, SARS Coronavirus
QRQKKQPTVT

SEQ ID NO: 82 - protein, 10 aa, SARS Coronavirus
QPTVTLLPAA

SEQ ID NO: 83 - protein, 17 aa, SARS Coronavirus
NGTITVEELKQLLEQWN

SEQ ID NO: 84 - protein, 10 aa, SARS Coronavirus
MADNGTITVE

SEQ ID NO: 85 - protein, 10 aa, SARS Coronavirus
TITVEELKQL

SEQ ID NO: 86 - protein, 10 aa, SARS Coronavirus
ELKQLLEQWN

SEQ ID NO: 87 - protein, 10 aa, SARS Coronavirus
LACFVLAAVY

SEQ ID NO: 88 - protein, 10 aa, SARS Coronavirus
RINWVTGGIA

SEQ ID NO: 89 - protein, 10 aa, SARS Coronavirus
TGGIAIAMAC

SEQ ID NO: 90 - protein, 10 aa, SARS Coronavirus
MYSFVSEETG

SEQ ID NO: 91 - protein, 10 aa, SARS Coronavirus
SEETGTLIVN

SEQ ID NO: 92 - protein, 10 aa, SARS Coronavirus
TLIVNSVLLF

SEQ ID NO: 93 - protein, 10 aa, SARS Coronavirus
MDLFMRFFTL

SEQ ID NO: 94 - protein, 10 aa, SARS Coronavirus
RFFTLGSITA

SEQ ID NO: 95 - protein, 10 aa, SARS Coronavirus
GSITAQPVKI

SEQ ID NO: 96 - protein, 10 aa, SARS Coronavirus
QPVKIDNASP

SEQ ID NO: 97 - protein, 10 aa, SARS Coronavirus
DNASPASTVH

SEQ ID NO: 98 - protein, 10 aa, SARS Coronavirus
ASTVHATATI

SEQ ID NO: 99 - protein, 10 aa, SARS Coronavirus
ATATIPLQAS

SEQ ID NO: 100 - protein, 10 aa, SARS Coronavirus
HLLLVAAGME

SEQ ID NO: 101 - protein, 10 aa, SARS Coronavirus
SVLLFLAFVV

SEQ ID NO: 102 - protein, 10 aa, SARS Coronavirus
LAFVVFLLVT

SEQ ID NO: 103 - protein, 10 aa, SARS Coronavirus
FLLVTLAILT

SEQ ID NO: 104 - protein, 10 aa, SARS Coronavirus
LAILTALRLC

SEQ ID NO: 105 - protein, 10 aa, SARS Coronavirus
ALRLCAYCCN

SEQ ID NO: 106 - protein, 10 aa, SARS Coronavirus
AYCCNIVNVS

SEQ ID NO: 107 - protein, 10 aa, SARS Coronavirus
IVNVSLVKPT

SEQ ID NO: 108 - protein, 10 aa, SARS Coronavirus
LVKPTVYVYS

SEQ ID NO: 109 - protein, 10 aa, SARS Coronavirus
VYVYSRVKNL

SEQ ID NO: 110 - protein, 10 aa, SARS Coronavirus
RVKNLNSSEG

SEQ ID NO: 111 - protein, 10 aa, SARS Coronavirus
SSEGVPDLLV

SEQ ID NO: 112 - protein, 10 aa, SARS Coronavirus
FVASFRLFAR

SEQ ID NO: 113 - protein, 10 aa, SARS Coronavirus
RLFARTRSMW

SEQ ID NO: 114 - protein, 10 aa, SARS Coronavirus
TRSMWSFNPE

SEQ ID NO: 115 - protein, 10 aa, SARS Coronavirus
SFNPETNILL

SEQ ID NO: 116 - protein, 10 aa, SARS Coronavirus
TNILLNVPLR

SEQ ID NO: 117 - protein, 10 aa, SARS Coronavirus
PKEITVATSR

SEQ ID NO: 118 - protein, 10 aa, SARS Coronavirus
VATSRTLSYY

SEQ ID NO: 119 - protein, 10 aa, SARS Coronavirus
TLSYYKLGAS

SEQ ID NO: 120 - protein, 10 aa, SARS Coronavirus
KLGASQRVGT

SEQ ID NO: 121 - protein, 10 aa, SARS Coronavirus
QRVGTDSGFA

SEQ ID NO: 122 - protein, 10 aa, SARS Coronavirus
DSGFAAYNRY

SEQ ID NO: 123 - protein, 10 aa, SARS Coronavirus
AYNRYRIGNY

SEQ ID NO: 124 - protein, 10 aa, SARS Coronavirus
QIQLSLLKVT

SEQ ID NO: 125 - protein, 10 aa, SARS Coronavirus
LLKVTAFQHQ

SEQ ID NO: 126 - protein, 10 aa, SARS Coronavirus
AFQHQNSKKT

SEQ ID NO: 127 - protein, 10 aa, SARS Coronavirus
NSKKTTKLVV

SEQ ID NO: 128 - protein, 10 aa, SARS Coronavirus
TKLVVILRIG

SEQ ID NO: 129 - protein, 10 aa, SARS Coronavirus
ILRIGTQVLK

SEQ ID NO: 130 - protein, 10 aa, SARS Coronavirus
TQVLKTMSLY

SEQ ID NO: 131 - protein, 10 aa, SARS Coronavirus
TMSLYMAISP

SEQ ID NO: 132 - protein, 10 aa, SARS Coronavirus
MAISPKFTTS

SEQ ID NO: 133 - protein, 10 aa, SARS Coronavirus
KFTTSLSLHK

SEQ ID NO: 134 - protein, 10 aa, SARS Coronavirus
LSLHKLLQTL

SEQ ID NO: 135 - protein, 10 aa, SARS Coronavirus
LLQTLVLKML

SEQ ID NO: 136 - protein, 10 aa, SARS Coronavirus
VLKMLHSSSL

SEQ ID NO: 137 - protein, 10 aa, SARS Coronavirus
HSSSLTSLLK

SEQ ID NO: 138 - protein, 10 aa, SARS Coronavirus
WIQFMMSRRR

SEQ ID NO: 139 - protein, 10 aa, SARS Coronavirus
MSRRRLLACL

SEQ ID NO: 140 - protein, 10 aa, SARS Coronavirus
LLACLCKHKK

SEQ ID NO: 141 - protein, 9 aa, SARS Coronavirus
KHKKVSTNL

SEQ ID NO: 142 - protein, 10 aa, SARS Coronavirus
STNLCTHSFR

SEQ ID NO: 143 - protein, 10 aa, SARS Coronavirus
MFHLVDFQVT

SEQ ID NO: 144 - protein, 10 aa, SARS Coronavirus
DFQVTIAEIL

SEQ ID NO: 145 - protein, 10 aa, SARS Coronavirus
IAEILIIIMR

SEQ ID NO: 146 - protein, 10 aa, SARS Coronavirus
IIIMRTFRIA

SEQ ID NO: 147 - protein, 10 aa, SARS Coronavirus
TFRIAIWNLD

SEQ ID NO: 148 - protein, 10 aa, SARS Coronavirus
IWNLDVIISS

SEQ ID NO: 149 - protein, 10 aa, SARS Coronavirus
VIISSIVRQL

SEQ ID NO: 150 - protein, 10 aa, SARS Coronavirus
IVRQLFKPLT

SEQ ID NO: 151 - protein, 10 aa, SARS Coronavirus
FKPLTKKNYS

SEQ ID NO: 152 - protein, 10 aa, SARS Coronavirus
KKNYSELDDE

SEQ ID NO: 153 - protein, 10 aa, SARS Coronavirus
DEEPMELDYP

SEQ ID NO: 154 - protein, 10 aa, SARS Coronavirus
GTRHTYQLRA

SEQ ID NO: 155 - protein, 10 aa, SARS Coronavirus
YQLRARSVSP

SEQ ID NO: 156 - protein, 10 aa, SARS Coronavirus
RSVSPKLFIR

SEQ ID NO: 157 - protein, 10 aa, SARS Coronavirus
KLFIRQEEVQ

SEQ ID NO: 158 - protein, 10 aa, SARS Coronavirus
QEEVQQELYS

SEQ ID NO: 159 - protein, 10 aa, SARS Coronavirus
QELYSPLFLI

SEQ ID NO: 160 - protein, 10 aa, SARS Coronavirus
PLFLIVAALV

SEQ ID NO: 161 - protein, 9 aa, SARS Coronavirus
AALVFLILC

SEQ ID NO: 162 - protein, 10 aa, SARS Coronavirus
FLILCFTIKR

SEQ ID NO: 163 - protein, 10 aa, SARS Coronavirus
MCLKILVRYN

SEQ ID NO: 164 - protein, 10 aa, SARS Coronavirus
LVRYNTRGNT

SEQ ID NO: 165 - protein, 10 aa, SARS Coronavirus
TRGNTYSTAW

SEQ ID NO: 166 - protein, 10 aa, SARS Coronavirus
YSTAWLCALG

SEQ ID NO: 167 - protein, 10 aa, SARS Coronavirus
LCALGKVLPF

SEQ ID NO: 168 - protein, 10 aa, SARS Coronavirus
KVLPFHRWHT

SEQ ID NO: 169 - protein, 10 aa, SARS Coronavirus
HRWHTMVQTC

SEQ ID NO: 170 - protein, 10 aa, SARS Coronavirus
MVQTCTPNVT

SEQ ID NO: 171 - protein, 10 aa, SARS Coronavirus
TPNVTINCQD

SEQ ID NO: 172 - protein, 10 aa, SARS Coronavirus
INCQDPAGGA

SEQ ID NO: 173 - protein, 10 aa, SARS Coronavirus
PAGGALIARC

SEQ ID NO: 174 - protein, 10 aa, SARS Coronavirus
LIARCWYLHE

SEQ ID NO: 175 - protein, 10 aa, SARS Coronavirus
WYLHEGHQTA

SEQ ID NO: 176 - protein, 10 aa, SARS Coronavirus
GHQTAAFRDV

SEQ ID NO: 177 - protein, 10 aa, SARS Coronavirus
AFRDVLVVLN

SEQ ID NO: 178 - protein, 10 aa, SARS Coronavirus
VLVVLNKRTN

SEQ ID NO: 179 - protein, 10 aa, SARS Coronavirus
NNTVYDPLQP

SEQ ID NO: 180 - protein, 10 aa, SARS Coronavirus
DPLQPELDSF

SEQ ID NO: 181 - protein, 10 aa, SARS Coronavirus
ELDSFKEELD

SEQ ID NO: 182 - protein, 10 aa, SARS Coronavirus
KEELDKYFKN

SEQ ID NO: 183 - protein, 10 aa, SARS Coronavirus
KYFKNHTSPD

SEQ ID NO: 184 - protein, 10 aa, SARS Coronavirus
HTSPDVDLGD

SEQ ID NO: 185 - protein, 10 aa, SARS Coronavirus
VDLGDISGIN

SEQ ID NO: 186 - protein, 10 aa, SARS Coronavirus
ISGINASVVN

SEQ ID NO: 187 - protein, 10 aa, SARS Coronavirus
ASVVNIQKEI

SEQ ID NO: 188 - protein, 10 aa, SARS Coronavirus
IQKEIDRLNE

SEQ ID NO: 189 - protein, 10 aa, SARS Coronavirus
DRLNEVAKNL

SEQ ID NO: 190 - protein, 10 aa, SARS Coronavirus
VAKNLNESLI

SEQ ID NO: 191 - protein, 10 aa, SARS Coronavirus
NESLIDLQEL

SEQ ID NO: 192 - protein, 10 aa, SARS Coronavirus
DLQELGKYEQ

SEQ ID NO: 193 - protein, 10 aa, SARS Coronavirus
GKYEQYIKWP

SEQ ID NO: 194 - protein, 10 aa, SARS Coronavirus
YIKWPWYVWL

SEQ ID NO: 195 - protein, 10 aa, SARS Coronavirus
GPDDQIGYYR